(19) United States
(12) Patent Application Publication
(10) Pub. No.: US 2026/0226503 A1
(43) Pub. Date: Aug. 6, 2026

(54) COMPOSITIONS AND METHODS FOR PURIFYING VIRAL VECTORS

(71) Applicant: North Carolina State University, Raleigh, NC (US)

(72) Inventors: Stefano Menegatti, Raleigh, NC (US); Wenning Chu, Raleigh, NC (US); Shriarjun Shastry, Raleigh, NC (US); Raphael Prodromou, Raleigh, NC (US); Eduardo Barbieri, Raleigh, NC (US)

(21) Appl. No.: 19/154,929

(22) PCT Filed: Feb. 9, 2024
(86) PCT No.: PCT/US2024/015225

§ 371 Date: Aug. 8, 2025

Related U.S. Application Data
(60) Provisional application No. 63/484,300, filed on Feb. 10, 2023.

(51) Int. Cl. C12N 15/86; B01D 15/38; B01J 20/26

(52) U.S. Cl. CPC C12N 15/86; B01D 15/3828; B01J 20/26; C12N 2750/14143; C12N 2750/14151

(57) ABSTRACT

The present disclosure provides materials and methods related to the purification of viral vectors. In particular, the present disclosure provides compositions, and related methods, comprising peptide ligands capable of removing process-related impurities (e.g., host cell proteins, nucleic acids, and media components) and product-related impurities (e.g., product fragments, product aggregates, and inactive forms derived from product degradation by or association with other species in the cell culture harvest) from biological fluids during the production and purification of adeno-associated viruses (AAVs).

### CROSS REFERENCE TO RELATED APPLICATIONS

This application claims priority to and the benefit of U.S. Provisional Patent Application No. 63/484,300 filed Feb. 10, 2023, which is incorporated herein by reference in its entirety and for all purposes.

### SEQUENCE LISTING STATEMENT

The contents of the electronic sequence listing titled (NCSU-41664-601; Size: 16,922 bytes; and Date of Creation: Feb. 9, 2024) is herein incorporated by reference in its entirety.

### FIELD

The present disclosure provides materials and methods related to the purification of viral vectors. In particular, the present disclosure provides compositions, and related methods, comprising peptide ligands capable of removing process-related impurities and product-related impurities from biological fluids during the production and purification of adeno-associated viruses (AAVs).

### BACKGROUND

Gene therapy provides a unique approach to cure inherited and acquired diseases by downregulating or replacing a defective gene with a functional one. As of 2022, almost 3,000 gene therapy clinical trials have been initiated and four gene therapy products approved by the Food and Drug Administration (FDA). A key role in the gene therapy revolution is played by viral vectors, owing to their ability to deliver a genomic payload efficiently and selectively to a target cell or tissue. While several classes of viral vectors are known and utilized today—employed in cell engineering (e.g., Lentivirus and Baculovirus) or vaccination and oncolytic applications (e.g., Adenovirus and Herpes Simplex Virus) —the field of gene therapy is dominated by Adeno-Associated Viruses (AAV) owing to their low toxicity/pathogenicity and efficient integration of the transgene into the host cells.

AAV is a small, non-enveloped icosahedral virus, whose capsid can pack a linear single-strand DNA (ssDNA) genome of up to about 5 kilobases. AAV capsids are formed by three virion proteins (VP1, VP2, and VP3), typically assembled in a 1:1:10 ratio. To date, 13 distinct AAV serotypes (AAV1-AAV13) are known—with the AAV2 being the most studied-which share a 65-99% sequence identity in their VPs and a 95-99% structural identity. The biomolecular variations among serotypes translate in specific cell/tissue tropism: cardiac, skeletal, and muscle cells are targeted by AAV1, AAV6, and AAV9; retina cells by AAV2 and AAV8; hepatocytes by AAV8, AAV9, and AAV-DJ; lung cells by AAV5, AAV6, and AAV9; cells in the central nervous system (CNS) by AAV1, AAV5, AAV6, AAV9, and AAV-rh10. Recently, recombinant AAVs (rAAVs) have been introduced, which feature improved gene packing and tissue tropism as well as lower immunogenicity and hepatotoxicity.

The manufacturing of AAVs relies on two expression systems, namely triple transfected human embryonic kidney (HEK 293) cells, which generate ~10¹⁴ vector genome-containing particles (vg) per liter of cell culture when harvested just 72 hours post-transfection and are ideal for serving small cohort of patients, such as those suffering from rare diseases; and the live baculovirus infection of Spodoptera frugiperda (Sf9) insect cells, which can be grown in serum-free media and avoid the replication of contaminating human agents, and are ideal for large AAV batches, such as those dedicated to fighting cancer and specific monogenic diseases.

The large number of particles needed for a single patient dosing, which can reach up to 10¹⁴ vg per kg of body weight, combined with stringent requirements of purity puts significant pressure on the downstream segment of the manufacturing process. The current platform process for AAV purification—reminiscent of the one established for mAbs—begins with an affinity-based capture step, which is tasked with removing most of the host cell proteins (HCPs) and DNA (hcDNA), and concentrating the AAV product for the subsequent steps of polishing and enrichment of full capsids. Current affinity adsorbents include chromatographic resins functionalized with heparin, whose applicability is limited to AAV2, or camelid single-domain antibodies. The latter include AVB Sepharose™ High Performance resin, which targets AAV serotypes 1, 2, 3, and 5; the POROS™ CaptureSelect™ AAVX affinity resin, which targets AAV1-AAV8, AAVrh10, and rAAVs; POROS™ CaptureSelect AAV8 and AAV9 resins, specific to AAV8 and AAV9; and AVIPure® AAV2, AAV8, and AAV9 affinity resins. Despite their excellent binding capacity (>10¹³ vp per mL of resin) and selectivity, these adsorbents feature high cost, low biochemical stability and short lifetime (<20 cycles), and require harsh elution conditions (pH<3.0) that can cause denaturation and aggregation of the AAV capsids, with consequent loss of transduction activity of the product.

### SUMMARY

Embodiments of the present disclosure include a composition for purifying an adeno-associated virus (AAV) from a biological fluid. In accordance with these embodiments, the composition comprises: (a) at least one peptide ligand that is at least six amino acids in length comprising the amino acid sequence X₁-X₂-X₃-X₄-X₅-X₆ (SEQ ID NO: 1), wherein X₁ is F, S, K, or W; X₂ is F, W, N, or K; X₃ is N, F, E, A, I, or W; X₄ is F, I, H, or K; X₅ is F, I, N, or W; and X₆ is K, F, I, S or A; and/or (b) at least one peptide ligand that is at least eight amino acids in length comprising the amino acid sequence X₁-X₂-X₃-X₄-X₅-X₆-X₇-X₈ (SEQ ID NO: 2), wherein X₁ is F, I, or N; X₂ is W, K, I, or A; X₃ is N, A, W, I, or F; X₄ is W, A, H, F, S or K; X₅ is H, F, A, I, or K; X₆ is H, F, A, or I; X₇ is F, K, W, S, or I; and X₈ is K, F, S, or I.

In some embodiments, the AAV is selected from the group consisting of AAV1, AAV2, AAV3, AAV4, AAV5, AAV6, AAV7, AAV8, AAV9, and AAVrh10. In some embodiments, the AAV is a recombinant AAV (rAAV).

In some embodiments, the at least one peptide ligand from (a) and/or (b) binds a site on the AAV capsid. In some embodiments, the binding site on the AAV capsid is located at an interface of VP1-VP2 virion proteins or at an interface of VP2-VP3 virion proteins. In some embodiments, the binding site on the AAV capsid is located at a conserved region.

In some embodiments, the at least one peptide ligand from (a) and/or (b) comprises an isoelectric point from about 6.5 to about 12.5.

In some embodiments, the at least one peptide ligand from (a) and/or (b) comprises a polarity value from about 5.0 to about 8.0.

In some embodiments, the at least one peptide ligand from (a) and/or (b) exhibits a disassociation constant (K_{D}) less than or equal to about 10⁻⁴ M. In some embodiments, the at least one peptide ligand from (a) and/or (b) exhibits a disassociation constant (K_{D}) higher than or equal to about 10⁻⁴ M at a pH that is at least 6.0.

In some embodiments, the at least one peptide ligand from (a) and/or (b) exhibits a dynamic binding capacity (DBC_{10%}) of at least 5×10¹² vp/mL of resin.

In some embodiments, the composition comprises at least one peptide from (a) and at least one peptide ligand from (b).

In some embodiments, the at least one peptide ligand from (a) comprises: (i) an amino acid sequence having at least 80% sequence identity with SEQ ID NO: 3; (ii) an amino acid sequence having at least 80% sequence identity with SEQ ID NO: 4; (iii) an amino acid sequence having at least 80% sequence identity with SEQ ID NO: 5; (iv) an amino acid sequence having at least 80% sequence identity with SEQ ID NO: 6; (v) an amino acid sequence having at least 80% sequence identity with SEQ ID NO: 7; (vi) an amino acid sequence having at least 80% sequence identity with SEQ ID NO: 8; (vii) an amino acid sequence having at least 80% sequence identity with SEQ ID NO: 9; (viii) an amino acid sequence having at least 80% sequence identity with SEQ ID NO: 10; and/or (ix) an amino acid sequence having at least 80% sequence identity with SEQ ID NO: 11.

In some embodiments, the at least one peptide ligand from (a) comprises: (i) an amino acid sequence comprising SEQ ID NO: 3; (ii) an amino acid sequence comprising SEQ ID NO: 4; (iii) an amino acid sequence comprising SEQ ID NO: 5; (iv) an amino acid sequence comprising SEQ ID NO: 6; (v) an amino acid sequence comprising SEQ ID NO: 7; (vi) an amino acid sequence comprising SEQ ID NO: 8; (vii) an amino acid sequence comprising SEQ ID NO: 9; (viii) an amino acid sequence comprising SEQ ID NO: 10; and/or (ix) an amino acid sequence comprising SEQ ID NO: 11.

In some embodiments, the composition comprises: (i) at least two peptide ligands selected from SEQ ID NOs: 3-11; (ii) at least three peptide ligands selected from SEQ ID NOs: 3-11; (iii) at least four peptide ligands selected from SEQ ID NOs: 3-11; (iv) at least five peptide ligands selected from SEQ ID NOs: 3-11; (v) at least six peptide ligands selected from SEQ ID NOs: 3-11; (vi) at least seven peptide ligands selected from SEQ ID NOs: 3-11; (vii) at least eight peptide ligands selected from SEQ ID NOs: 3-11; or (viii) all nine peptide ligands of SEQ ID NOs: 3-11.

In some embodiments, the at least one peptide ligand from (b) comprises: (i) an amino acid sequence having at least 80% sequence identity with SEQ ID NO: 12; (ii) an amino acid sequence having at least 80% sequence identity with SEQ ID NO: 13; (iii) an amino acid sequence having at least 80% sequence identity with SEQ ID NO: 14; (iv) an amino acid sequence having at least 80% sequence identity with SEQ ID NO: 15; (v) an amino acid sequence having at least 80% sequence identity with SEQ ID NO: 16; (vi) an amino acid sequence having at least 80% sequence identity with SEQ ID NO: 17; (vii) an amino acid sequence having at least 80% sequence identity with SEQ ID NO: 18; (viii) an amino acid sequence having at least 80% sequence identity with SEQ ID NO: 19; and/or (ix) an amino acid sequence having at least 80% sequence identity with SEQ ID NO: 20.

In some embodiments, the at least one peptide ligand from (b) comprises: (i) an amino acid sequence comprising SEQ ID NO: 12; (ii) an amino acid sequence comprising SEQ ID NO: 13; (iii) an amino acid sequence comprising SEQ ID NO: 14; (iv) an amino acid sequence comprising SEQ ID NO: 15; (v) an amino acid sequence comprising SEQ ID NO: 16; (vi) an amino acid sequence comprising SEQ ID NO: 17; (vii) an amino acid sequence comprising SEQ ID NO: 18; (viii) an amino acid sequence comprising SEQ ID NO: 19; and/or (ix) an amino acid sequence comprising SEQ ID NO: 20.

In some embodiments, the composition comprises: (i) at least two peptide ligands selected from SEQ ID NOs: 12-20; (ii) at least three peptide ligands selected from SEQ ID NOs: 12-20; (iii) at least four peptide ligands selected from SEQ ID NOs: 12-20; (iv) at least five peptide ligands selected from SEQ ID NOs: 12-20; (v) at least six peptide ligands selected from SEQ ID NOs: 12-20; (vi) at least seven peptide ligands selected from SEQ ID NOs: 12-20; (vii) at least eight peptide ligands selected from SEQ ID NOs: 12-20; or (viii) all nine peptide ligands of SEQ ID NOs: 12-20.

In some embodiments, the at least one peptide ligand comprises a linker. In some embodiments, the linker is bound to the C-terminus of the peptide ligand, and wherein the linker comprises a Glyₙ or a [Gly-Ser-Gly]ₘ, wherein 6≥n≥1 and 3≥m≥1.

In some embodiments, the at least one peptide ligand is bound to a solid support. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate, polyolefin, polyester, polysaccharide, iron oxide, silica, titania, and/or zirconia.

In some embodiments, the biological fluid is a cell culture fluid. In some embodiments, the biological fluid comprises a supernatant and/or a cellular lysate.

In some embodiments, the biological fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof.

In some embodiments, the biological fluid is derived from HEK cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof.

In some embodiments, the biological fluid is derived from a virus production cell line. In some embodiments, the virus production cell line is selected from the group consisting of MDCK-S, MDCK-A, Vero cells, LLC-MK2D, PER.C6, EB66, and AGE1.CR cells, Spodoptera frugiperda (Sf9) cells, and HeLa cells, or any derivatives or variants thereof.

Embodiments of the present disclosure also include an adsorbent comprising any of the compositions described herein.

Embodiments of the present disclosure also include a method of purifying an adeno-associated virus (AAV) from a biological fluid. In accordance with these embodiments, the method includes contacting a composition comprising any of the peptide ligands described herein, or an adsorbent comprising any of the peptides described herein, with a biological fluid comprising the AAV, wherein the at least one peptide ligand binds the AAV, and eluting the AAV from the peptide ligand.

In some embodiments, the elution is performed at pH from about 6.0 to about 7.5.

In some embodiments, the method further comprises a washing step before eluting the AAV from the peptide ligand.

In some embodiments, the method results in at least a 50% yield for the AAV. In some embodiments, the method produces at least an 80-fold reduction in host cell proteins.

Embodiments of the present disclosure also include an adeno-associated virus (AAV) purified using any of the methods described herein. In some embodiments, the AAV exhibits at least 50% transduction activity.

### BRIEF DESCRIPTION OF THE DRAWINGS

FIG. 1: Process for identification of AAV-targeting peptide ligands. An ensemble of 6-mer or 8-mer peptide-ChemMatrix beads are (1) collectively incubated with a screening mix comprising AF594-labeled AAV2 (red) at 5·10¹¹ vp/mL and AF488-labeled HEK 293 HCPs (green) at ~0.5 mg/mL; (2) the beads are fed to a microfluidic bead sorting device, which discards all non-fluorescent, the green-only, and red-and-green beads, and retains every red-only bead; (3) the latter is exposed to an elution buffer comprising 1 M MgCl₂ in 20 mM Bis-Tris buffer at pH 6.0 for 2 mins at room temperature; (4) every bead that displays at least a 10-fold loss of red fluorescence is selected as a positive lead; (5) finally, positive beads are analyzed by Edman degradation to identify the candidate AAV-targeting peptides. (B) Sequence homology of the selected 6-mer and 8-mer peptides prepared using Weblogo.

FIGS. 2A-2B: Values of loss (orange, calculated as the ratio of the AAV titer in the flow-through and wash fractions vs. load) and yield (green, calculated as the ratio of the AAV titer in the elution fraction vs. load) of (A) AAV2 and (B) AAV9 obtained via bind-and-elute studies in non-competitive mode using peptide-based resins KFNHWFG- (W1), WKAHNKG- (W2), IWWHIAKFG- (W3), FWNWHHFKG- (W4), FWWAAFFKG- (W5), IAFKKISIG- (W6), IKIFFFFSG- (W7), KWWIWAG- (W8), WWIKISG- (W9), FFNFFKG- (W10), FNHFFIG- (W11), GYISRHPG- (W12) Toyopearl resins, and control adsorbents POROS™ CaptureSelect™ AAVX Affinity and AVB Sepharose HP resins. The AAV titer in the flow-through, wash, and elution fractions was measured using serotype-specific ELISA kits.

FIGS. 3A-3D: Representative complexes formed by peptides KFNHWFG (green), FFNFFKG (yellow), FNHFFIG (pink), IWWHIAKFG (brown), FWNWHHFKG (cyan), and FWWAAFFKG (orange) with the capsids of (A) AAV2 (PDB IDs 5IPI, 6IH9, 6IHB, and 6U0V); (B) AAV6 (3SHM, 3OAH, 4V86, and 5EGC); (C) AAV8 (2QA0, 3RAA, 6PWA, 6U2V, and 6V10); and (D) AAV9 (3UX1, 7MT0, 7WJW, and 7WJX) obtained via molecular docking and dynamics simulations. The segments of the VP that are not solvent accessible or whose homology among AAV serotypes is lower than 95% are in grey, the homologous segments of VP that are solvent accessible and displayed on the concave side of the capsid are in pink, and the homologous segments of VP that are solvent accessible and displayed on the convex side of the capsid are in red; the binding sites are labeled in FIG. 6.

FIGS. 4A-4B: Values of loss (orange, calculated as the ratio of the AAV titer in the flow-through and wash fractions vs. load) and yield (green, calculated as the ratio of the AAV titer in the elution fraction vs. load) of AAV2 and logarithmic reduction of HCPs (HCP LRV, red triangles) obtained via chromatographic purification of AAV2 from a clarified HEK 293 cell lysate (AAV2 titer: ~1.6·10¹¹ vp/mL; HCP titer: ~0.5 mg/mL) using (A) control adsorbents POROS™ CaptureSelect™ AAVX Affinity and AVB Sepharose HP resins as well as peptide-based resins KFNHWFG- (W1), WKAHNKG- (W2), IWWHIAKFG- (W3), FWNWHHFKG- (W4), FWWAAFFKG- (W5), (B) IAFKKISIG- (W6), IKIFFFFSG- (W7), KWWIWAG- (W8), WWIKISG- (W9), FFNFFKG- (W10), FNHFFIG- (W11), GYISRHPG- (W12) Toyopearl resins. The AAV titer in the flow-through, wash, and elution fractions was measured using serotype-specific ELISA kits.

FIG. 5: Values of relative transduction efficiency of AAV2 purified from a clarified HEK 293 cell lysate using peptide-based adsorbents FWWAAFFKG- (W5), IAFKKISIG- (W6), IKIFFFFSG- (W7), KWWIWAG- (W8), WWIKISG- (W9), FFNFFKG- (W10), FNHFFIG- (W11), and GYISRHPG- (W12) Toyopearl resins. The transduction efficiency (TU/vp) of eluted AAV2 was measured on human epithelial (HT1080) by performing a green fluorescence assay using a CytoFLEX Flow Cytometer. The values of relative transduction efficiency were calculated as the ratio of transduction efficiency of the AAV2 eluted from a peptide-based adsorbent vs. the transduction efficiency of the AAV2 eluted from POROS™ CaptureSelect™ AAVX Affinity resin.

FIGS. 6A-6C: Druggability study of AAV virion protein (VP). (A) Structure of the VP1 from AAV1 (PDB ID: 6JCR), AAV2 (6IH9), AAV3 (3KIC), AAV4 (2G8G), AAV5 (7KP3), AAV6 (5EGC), AAV7 (7JOT), AAV8 (2QA0), and AAV9 (7WJX); the segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon; (B) putative binding sites 1-5 identified on the homologous segments of VP that are solvent accessible and displayed on the convex side of the AAV2 capsid; and (C) putative binding sites 1-6 identified on the homologous segments of VP that are solvent accessible and displayed on the convex side of the AAV9 capsid.

FIGS. 7A-7C: (A)-(C) Chromatograms of AAV2 binding and elution using peptide-based adsorbents KFNHWFG- (W1), WKAHNKG- (W2), IWWHIAKFG- (W3), FWNWHHFKG- (W4), FWWAAFFKG- (W5), IAFKKISIG- (W6), IKIFFFFSG- (W7), KWWIWAG- (W8), WWIKISG- (W9), FFNFFKG- (W10), FNHFFIG- (W11), GYISRHPG- (W12) Toyopearl resins, and control adsorbents POROS™ CaptureSelect™ AAVX Affinity and AVB Sepharose HP resins. Binding was conducted in 20 mM NaCl in 20 mM Bis-Tris buffer at pH 7.0 (RT: 3 min); elution from the peptide-functionalized resins was conducted using 1M MgCl₂ in 20 mM Bis-Tris buffer at pH 6.0 (RT: 2 min); elution from POROS™ CaptureSelect™ AAVX Affinity resin and AVB Sepharose HP resin was conducted using 0.2 M MgCl₂ in 200 mM citrate buffer at pH 2.2 and PBS at pH 2.0, respectively (RT: 2 min). (D) SDS-PAGE analysis (reducing condition, silver staining) of the elution fractions; labels: MW, molecular weight marker; AAV2 standard; VP, virion proteins; E, eluted fraction.

FIGS. 8A-8C: (A)-(C) Chromatograms of AAV9 binding and elution using peptide-based adsorbents KFNHWFG- (W1), WKAHNKG- (W2), IWWHIAKFG- (W3), FWNWHHFKG- (W4), FWWAAFFKG- (W5), IAFKKISIG- (W6), IKIFFFFSG- (W7), KWWIWAG- (W8), WWIKISG- (W9), FFNFFKG- (W10), FNHFFIG- (W11), GYISRHPG- (W12) Toyopearl resins, and control adsorbents POROS™ CaptureSelect™ AAVX Affinity and AVB Sepharose HP resins. Binding was conducted in 20 mM NaCl in 20 mM Bis-Tris buffer at pH 7.0 (RT: 3 min); elution from the peptide-functionalized resins was conducted using 1M MgCl₂ in 20 mM Bis-Tris buffer at pH 6.0 (RT: 2 min); elution from POROS™ CaptureSelect™ AAVX Affinity resin and AVB Sepharose HP resin was conducted using 0.2 M MgCl₂ in 200 mM citrate buffer at pH 2.2 and PBS at pH 2.0, respectively (RT: 2 min). (D) SDS-PAGE analysis (reducing condition, silver staining) of the elution fractions; labels: MW, molecular weight marker; AAV9 standard; VP, virion proteins; E, eluted fraction.

FIGS. 9A-9B: Complexes formed by peptide KFNHWF-GSG with the solvent accessible peptide segments displayed on the convex side of the VP1-VP2-VP3 cluster of AAV2 (PDB IDs: 6U0V, 6IH9, 5IPI, and 6IHB) and AAV9 (3UX1, 7MT0, 7WJW, and 7WJX). The segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon; the binding sites are labeled in FIG. 6; the peptide ligands are in blue cartoon.

FIGS. 10A-10B: Complexes formed by peptide IWWHIAKF-GSG with the solvent accessible peptide segments displayed on the convex side of the VP1-VP2-VP3 cluster of AAV2 (PDB IDs: 6U0V, 6IH9, 5IPI, and 6IHB) and AAV9 (3UX1, 7MT0, 7WJW, and 7WJX). The segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon; the binding sites are labeled in FIG. 6; the peptide ligands are in blue cartoon.

FIGS. 11A-11B: Complexes formed by peptide FWWAAFFK-GSG with the solvent accessible peptide segments displayed on the convex side of the VP1-VP2-VP3 cluster of AAV2 (PDB IDs: 6U0V, 6IH9, 5IPI, and 6IHB) and AAV9 (3UX1, 7MT0, 7WJW, and 7WJX). The segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon; the binding sites are labeled in FIG. 6; the peptide ligands are in blue cartoon.

FIGS. 12A-12B: Complexes formed by peptide FWNWHHFK-GSG with the solvent accessible peptide segments displayed on the convex side of the VP1-VP2-VP3 cluster of AAV2 (PDB IDs: 6U0V, 6IH9, 5IPI, and 6IHB) and AAV9 (3UX1, 7MT0, 7WJW, and 7WJX). The segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon; the binding sites are labeled in FIG. 6; the peptide ligands are in blue cartoon.

FIGS. 13A-13B: Complexes formed by peptide FNHFFI-GSG with the solvent accessible peptide segments displayed on the convex side of the VP1-VP2-VP3 cluster of AAV2 (PDB IDs: 6U0V, 6IH9, 5IPI, and 6IHB) and AAV9 (3UX1, 7MT0, 7WJW, and 7WJX). The segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon; the binding sites are labeled in FIG. 6; the peptide ligands are in blue cartoon.

FIGS. 14A-14B: Complexes formed by peptide FFNFFK-GSG with the solvent accessible peptide segments displayed on the convex side of the VP1-VP2-VP3 cluster of AAV2 (PDB IDs: 6U0V, 6IH9, 5IPI, and 6IHB) and AAV9 (3UX1, 7MT0, 7WJW, and 7WJX). The segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon; the binding sites are labeled in FIG. 6; the peptide ligands are in blue cartoon.

FIGS. 15A-15B: (A)-(B) Chromatograms of AAV2 purification from a clarified HEK 293 cell lysate (AAV2 titer: ~1.6·10¹¹ vp/mL; HCP titer: ~0.5 mg/mL) using peptide-based adsorbents KFNHWFG- (W1), WKAHNKG- (W2), IWWHIAKFG- (W3), FWNWHHFKG- (W4), FWWAAFFKG- (W5), IAFKKISIG- (W6), IKIFFFFSG- (W7), KWWIWAG- (W8), WWIKISG- (W9), FFNFFKG- (W10), FNHFFIG- (W11), GYISRHPG- (W12) Toyopearl resins, and control adsorbents POROS™ CaptureSelect™ AAVX Affinity and AVB Sepharose HP resins. Binding was conducted in 20 mM NaCl in 20 mM Bis-Tris buffer at pH 7.0 (RT: 3 min); elution from the peptide-functionalized resins was conducted using 1M MgCl₂ in 20 mM Bis-Tris buffer at pH 6.0 (RT: 2 min); elution from POROS™ CaptureSelect™ AAVX Affinity resin and AVB Sepharose HP resin was conducted using 0.2 M MgCl₂ in 200 mM citrate buffer at pH 2.2 and PBS at pH 2.0, respectively (RT: 2 min).

FIGS. 16A-16O: Size Exclusion Chromatography (SEC) analysis of (A) a clarified HEK 293 cell lysate (AAV2 titer: ~1.6·10¹¹ vp/mL; HCP titer: ~0.5 mg/mL) and the elution fractions obtained from the purification of AAV2 from the clarified lysate using control (B) POROS™ CaptureSelect™ AAVX Affinity resin and (C) AVB Sepharose HP resin as well as peptide-based adsorbents (D) KFNHWFG-, (E) WKAHNKG-, (F) IWWHIAKFG-, (G) FWNWHHFKG-, (H) FWWAAFFKG, (I) IAFKKISIG-, (J) IKIFFFFSG-, (K) KWWIWAG-, (L) WWIKISG-, (M) FFNFFKG-, (N) FNHFFIG-, and (O) GYISRHPG-Toyopearl resins.

FIGS. 17A-17O: Steric Exclusion Chromatography (SXC) analysis of (A) a clarified HEK 293 cell lysate (AAV2 titer: ~1.6·10¹¹ vp/mL; HCP titer: ~0.5 mg/mL) and the elution fractions obtained from the purification of AAV2 from the clarified lysate using control (B) POROS™ CaptureSelect™ AAVX Affinity resin and (C) AVB Sepharose HP resin as well as peptide-based adsorbents (D) KFNHWFG-, (E) WKAHNKG-, (F) IWWHIAKFG-, (G) FWNWHHFKG-, (H) FWWAAFFKG, (I) IAFKKISIG-, (J) IKIFFFFSG-, (K) KWWIWAG-, (L) WWIKISG-, (M) FFNFFKG-, (N) FNHFFIG-, and (O) GYISRHPG-Toyopearl resins.

FIG. 18: Breakthrough curves of AAV2 obtained by loading a clarified HEK 293 cell lysate (AAV2 titer: ~3.2·10¹¹ vp/mL; HCP titer: ~0.5 mg/mL) on adsorbents KFNHWFG-, IWWHIAKFG-, FWNWHHFKG-, IKIFFFFSG-, and FFNFFKG-Toyopearl resins at residence time (RT) of 3 mins.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “sequence identity” generally refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “target” or “target biologic” generally refers to a target protein, peptide, polypeptide, nucleic acid, ribonucleoprotein complex, nucleic acid construct, supramolecular construct, virus, viral construct, virus-like particle, cell, organelle, small molecule, and any combinations thereof, which may be present in a sample (e.g., biological fluid) comprising one or more process-related impurities and/or product-related substances. In some embodiments, the target or target biologic is an antibody or any antigen binding fragment/derivative thereof (e.g., monoclonal or polyclonal antibody). In other embodiments, the target or target biologic is a viral vector (e.g., AAV).

As used herein, the term “host cell protein” or “HCP” refers to any protein produced or encoded by the organism used to produce a recombinant polypeptide product and unrelated to the intended product. HCPs are generally undesirable in the final drug substance.

As used herein, a “mixture” comprises a target biologic of interest (for which purification is desired) and one or more contaminant or impurity. In some embodiments, the mixture is produced from a host cell or organism that expresses the protein of interest (either naturally or recombinantly). Such mixtures include, for example, cell cultures, cell lysates, and clarified bulk (e.g., clarified cell culture supernatant).

As used herein, a “derivative” with respect to a peptide or polypeptide generally has the amino acid sequence of a reference peptide or variant, but additionally comprises a chemical modification of one or more of its amino acid side groups, α-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine ε-amino groups, N-alkylation of arginine, histidine, or lysine, alkylation of glutamic or aspartic carboxylic acid groups, and deamidation of glutamine or asparagine. Modifications of the terminal amino include, without limitation, the desamino, N-lower alkyl, N-di-lower alkyl, constrained alkyls (e.g., branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily-skilled peptide chemist. The α-carbon of an amino acid may be mono- or dimethylated.

Additionally, as used herein, a “variant” with respect to a peptide or polypeptide generally refers to a peptide or polypeptide whose base amino acid sequence was derived from that of a reference peptide or polypeptide. A variant can include conservative and/or non-conservative amino acid substitutions (including non-natural amino acids and L and D forms).

### 2. Compositions and Methods for Removing Process and Product-Related Impurities

a. Compositions

In seeking robust alternatives to protein ligands, an ensemble of synthetic peptides was developed that bind AAVs selectively, enable their elution under near-physiological conditions, and can be reused multiple times without losing binding strength and selectivity. The peptide ligands described in the present disclosure target conserved binding sites found in all AAV serotypes (i) via multi-site interactions, which provide the necessary binding strength and capacity for effective product capture, although (ii) the single AAV:peptide complexes can be easily dissociated, thus enabling high product recovery at mild elution conditions.

Adeno-associated viruses (AAVs) are the vector of choice for delivering gene therapies that can cure inherited and acquired diseases. Clinical research on various AAV serotypes significantly increased in recent years alongside regulatory approvals of AAV-based therapies. The current AAV purification platform hinges on the capture step, for which several affinity resins are commercially available. These adsorbents rely on protein ligands—typically camelid antibodies—that provide high binding capacity and selectivity, but suffer from low biochemical stability and high cost, and impose harsh elution conditions (pH<3) that can harm the transduction activity of recovered AAVs. Addressing these challenges, this study introduces peptide ligands that selectively capture AAVs and release them under mild conditions (pH 6.0). The peptide sequences were identified by screening a focused library and modeled in silico against AAV serotypes 2 and 9 (AAV2 and AAV9) to select candidate ligands that target homologous sites at the interface of the VP1-VP2 and VP2-VP3 virion proteins with mild binding strength (K_{D}~10⁻⁵, 10⁻⁶ M). Selected peptides were conjugated to Toyopearl resin and evaluated via binding studies against AAV2 and AAV9, demonstrating the ability to target both serotypes with values of dynamic binding capacity (DBC_{10%}>10¹³ vp per mL of resin) and product yields (~50-80%) on par with commercial adsorbents. The peptide-based adsorbents were finally utilized to purify AAV2 from a HEK 293 cell lysate, affording high recovery (50-80%), 80-to-400-fold reduction of host cell proteins (HCPs), and high transduction activity (up to 80%) of the purified viruses.

In accordance with these embodiments, the present disclosure provides compositions and methods for purifying an adeno-associated virus (AAV) from a biological fluid. In some embodiments, the composition comprises: (a) at least one peptide ligand that is at least six amino acids in length comprising the amino acid sequence X₁-X₂-X₃-X₄-X₅-X₆ (SEQ ID NO: 1), wherein X₁ is F, S, K, or W; X₂ is F, W, N, or K; X₃ is N, F, E, A, I, or W; X₄ is F, I, H, or K; X₅ is F, I, N, or W; and X₆ is K, F, I, S or A; and/or (b) at least one peptide ligand that is at least eight amino acids in length comprising the amino acid sequence X₁-X₂-X₃-X₄-X₅-X₆-X₇-X₈ (SEQ ID NO: 2), wherein X₁ is F, I, or N; X₂ is W, K, I, or A; X₃ is N, A, W, I, or F; X₄ is W, A, H, F, S or K; X₅ is H, F, A, I, or K; X₆ is H, F, A, or I; X₇ is F, K, W, S, or I; and X₈ is K, F, S, or I.

In some embodiments, the AAV is selected from the group consisting of AAV1, AAV2, AAV3, AAV4, AAV5, AAV6, AAV7, AAV8, AAV9, and AAVrh10. In some embodiments, the AAV is a recombinant AAV (rAAV).

In some embodiments, at least one peptide ligand described herein is capable of binding a site on the AAV capsid. In some embodiments, the binding site on the AAV capsid is located at an interface of VP1-VP2 virion proteins. In some embodiments, the binding site on the AAV capsid is located at an interface of VP2-VP3 virion proteins. In some embodiments, the binding site on the AAV capsid is located in a conserved region.

In some embodiments, a peptide ligand of the present disclosure comprises an isoelectric point from about 6.5 to about 12.5. In some embodiments, a peptide ligand of the present disclosure comprises an isoelectric point from about 7.0 to about 12.5. In some embodiments, a peptide ligand of the present disclosure comprises an isoelectric point from about 7.5 to about 12.5. In some embodiments, a peptide ligand of the present disclosure comprises an isoelectric point from about 8.0 to about 12.5. In some embodiments, a peptide ligand of the present disclosure comprises an isoelectric point from about 8.5 to about 12.5. In some embodiments, a peptide ligand of the present disclosure comprises an isoelectric point from about 9.0 to about 12.5. In some embodiments, a peptide ligand of the present disclosure comprises an isoelectric point from about 10.0 to about 12.5. In some embodiments, a peptide ligand of the present disclosure comprises an isoelectric point from about 10.5 to about 12.5. In some embodiments, a peptide ligand of the present disclosure comprises an isoelectric point from about 11.0 to about 12.5. In some embodiments, a peptide ligand of the present disclosure comprises an isoelectric point from about 11.5 to about 12.5. In some embodiments, a peptide ligand of the present disclosure comprises an isoelectric point from about 12.0 to about 12.5. In some embodiments, a peptide ligand of the present disclosure comprises an isoelectric point from about 6.5 to about 12.0. In some embodiments, a peptide ligand of the present disclosure comprises an isoelectric point from about 6.5 to about 11.5. In some embodiments, a peptide ligand of the present disclosure comprises an isoelectric point from about 6.5 to about 11.0. In some embodiments, a peptide ligand of the present disclosure comprises an isoelectric point from about 6.5 to about 10.5. In some embodiments, a peptide ligand of the present disclosure comprises an isoelectric point from about 6.5 to about 10.0. In some embodiments, a peptide ligand of the present disclosure comprises an isoelectric point from about 6.5 to about 9.5. In some embodiments, a peptide ligand of the present disclosure comprises an isoelectric point from about 6.5 to about 9.0. In some embodiments, a peptide ligand of the present disclosure comprises an isoelectric point from about 6.5 to about 8.5. In some embodiments, a peptide ligand of the present disclosure comprises an isoelectric point from about 6.5 to about 8.0. In some embodiments, a peptide ligand of the present disclosure comprises an isoelectric point from about 6.5 to about 7.5. In some embodiments, a peptide ligand of the present disclosure comprises an isoelectric point from about 6.5 to about 7.0.

In some embodiments, a peptide ligand of the present disclosure comprises a polarity value from about 5.0 to about 8.0. In some embodiments, a peptide ligand of the present disclosure comprises a polarity value from about 5.0 to about 7.5. In some embodiments, a peptide ligand of a present disclosure comprises a polarity value from about 5.0 to about 6.0. In some embodiments, a peptide ligand of the present disclosure comprises a polarity value from about 5.0 to about 5.5. In some embodiments, a peptide ligand of the present disclosure comprises a polarity value from about 5.5 to about 8.0. In some embodiments, a peptide ligand of the present disclosure comprises a polarity value from about 6.0 to about 8.0. In some embodiments, a peptide ligand of the present disclosure comprises a polarity value from about 6.5 to about 8.0. In some embodiments, a peptide ligand of the present disclosure comprises a polarity value from about 7.0 to about 8.0. In some embodiments, a peptide ligand of the present disclosure comprises a polarity value from about 7.5 to about 8.0.

In some embodiments, a peptide ligand from the present disclosure exhibits a disassociation constant (K_{D}) for an AAV that is less than or equal to about 10⁻³ M. In some embodiments, a peptide ligand from the present disclosure exhibits a disassociation constant (K_{D}) less than or equal to about 10⁻⁴ M. In some embodiments, a peptide ligand from the present disclosure exhibits a disassociation constant (K_{D}) less than or equal to about 10⁻⁵ M. In some embodiments, a peptide ligand from the present disclosure exhibits a disassociation constant (K_{D}) less than or equal to about 10⁻⁶ M. In some embodiments, a peptide ligand from the present disclosure exhibits a disassociation constant (K_{D}) less than or equal to about 10⁻⁷ M. In some embodiments, a peptide ligand from the present disclosure exhibits a disassociation constant (K_{D}) less than or equal to about 10⁻⁸ M. In some embodiments, a peptide ligand from the present disclosure exhibits a disassociation constant (K_{D}) less than or equal to about 10⁻⁹ M. In some embodiments, a peptide ligand from the present disclosure exhibits a disassociation constant (K_{D}) less than or equal to about 10⁻¹⁰ M. In some embodiments, a peptide ligand from the present disclosure exhibits a disassociation constant (K_{D}) from about 10⁻³ M to about 10⁻¹⁰ M. In some embodiments, a peptide ligand from the present disclosure exhibits a disassociation constant (K_{D}) from about 10⁻⁴ M to about 10⁻⁸ M. In some embodiments, a peptide ligand from the present disclosure exhibits a disassociation constant (K_{D}) from about 10⁻⁵ M to about 10⁻⁷ M. In some embodiments, a peptide ligand from the present disclosure exhibits a disassociation constant (K_{D}) from about 10⁻⁶ M to about 10⁻⁸ M.

In some embodiments, a peptide ligand from the present disclosure exhibits a dynamic binding capacity (DBC_{10%}) of at least 10¹² vp per mL of resin. In some embodiments, a peptide ligand from the present disclosure exhibits a dynamic binding capacity (DBC_{10%}) of at least 10¹³ vp per mL of resin. In some embodiments, a peptide ligand from the present disclosure exhibits a dynamic binding capacity (DBC_{10%}) of at least 10¹⁴ vp per mL of resin. In some embodiments, a peptide ligand from the present disclosure exhibits a dynamic binding capacity (DBC_{10%}) of at least 10¹⁵ vp per mL of resin. In some embodiments, a peptide ligand from the present disclosure exhibits a dynamic binding capacity (DBC_{10%}) of at least 10¹⁶ vp per mL of resin. In some embodiments, a peptide ligand from the present disclosure exhibits a dynamic binding capacity (DBC_{10%}) of at least 10¹⁷ vp per mL of resin. In some embodiments, a peptide ligand from the present disclosure exhibits a dynamic binding capacity (DBC_{10%}) of at least 10¹⁸ vp per mL of resin. In some embodiments, a peptide ligand from the present disclosure exhibits a dynamic binding capacity (DBC_{10%}) of at least 10¹⁹ vp per mL of resin. In some embodiments, a peptide ligand from the present disclosure exhibits a dynamic binding capacity (DBC_{10%}) of at least 10²⁰ vp per mL of resin. In some embodiments, a peptide ligand from the present disclosure exhibits a dynamic binding capacity (DBC_{10%}) of about 10¹² vp per mL of resin to about 10²⁰ vp per mL of resin. In some embodiments, a peptide ligand from the present disclosure exhibits a dynamic binding capacity (DBC_{10%}) of about 10¹² vp per mL of resin to about 10¹⁸ vp per mL of resin. In some embodiments, a peptide ligand from the present disclosure exhibits a dynamic binding capacity (DBC_{10%}) of about 10¹² vp per mL of resin to about 10¹⁵ vp per mL of resin.

In some embodiments, the compositions of the present disclosure include at least one peptide that is at least six amino acids in length comprising the amino acid sequence X₁-X₂-X₃-X₄-X₅-X₆ (SEQ ID NO: 1), wherein X₁ is F, S, K, or W; X₂ is F, W, N, or K; X₃ is N, F, E, A, I, or W; X₄ is F, I, H, or K; X₅ is F, I, N, or W; and X₆ is K, F, I, S or A; and at least one peptide ligand that is at least eight amino acids in length comprising the amino acid sequence X₁-X₂-X₃-X₄-X₅-X₆-X₇-X₈ (SEQ ID NO: 2), wherein X₁ is F, I, or N; X₂ is W, K, I, or A; X₃ is N, A, W, I, or F; X₄ is W, A, H, F, S or K; X₅ is H, F, A, I, or K; X₆ is H, F, A, or I; X₇ is F, K, W, S, or I; and X₈ is K, F, S, or I.

In some embodiments, a peptide ligand from the present disclosure comprises: (i) an amino acid sequence having at least 80% sequence identity with SEQ ID NO: 3; (ii) an amino acid sequence having at least 80% sequence identity with SEQ ID NO: 4; (iii) an amino acid sequence having at least 80% sequence identity with SEQ ID NO: 5; (iv) an amino acid sequence having at least 80% sequence identity with SEQ ID NO: 6; (v) an amino acid sequence having at least 80% sequence identity with SEQ ID NO: 7; (vi) an amino acid sequence having at least 80% sequence identity with SEQ ID NO: 8; (vii) an amino acid sequence having at least 80% sequence identity with SEQ ID NO: 9; (viii) an amino acid sequence having at least 80% sequence identity with SEQ ID NO: 10; and/or (ix) an amino acid sequence having at least 80% sequence identity with SEQ ID NO: 11.

In some embodiments, a peptide ligand from the present disclosure comprises: (i) an amino acid sequence having at least 85% sequence identity with SEQ ID NO: 3; (ii) an amino acid sequence having at least 85% sequence identity with SEQ ID NO: 4; (iii) an amino acid sequence having at least 85% sequence identity with SEQ ID NO: 5; (iv) an amino acid sequence having at least 85% sequence identity with SEQ ID NO: 6; (v) an amino acid sequence having at least 85% sequence identity with SEQ ID NO: 7; (vi) an amino acid sequence having at least 85% sequence identity with SEQ ID NO: 8; (vii) an amino acid sequence having at least 85% sequence identity with SEQ ID NO: 9; (viii) an amino acid sequence having at least 85% sequence identity with SEQ ID NO: 10; and/or (ix) an amino acid sequence having at least 85% sequence identity with SEQ ID NO: 11.

In some embodiments, a peptide ligand from the present disclosure comprises: (i) an amino acid sequence having at least 90% sequence identity with SEQ ID NO: 3; (ii) an amino acid sequence having at least 90% sequence identity with SEQ ID NO: 4; (iii) an amino acid sequence having at least 90% sequence identity with SEQ ID NO: 5; (iv) an amino acid sequence having at least 90% sequence identity with SEQ ID NO: 6; (v) an amino acid sequence having at least 90% sequence identity with SEQ ID NO: 7; (vi) an amino acid sequence having at least 90% sequence identity with SEQ ID NO: 8; (vii) an amino acid sequence having at least 90% sequence identity with SEQ ID NO: 9; (viii) an amino acid sequence having at least 90% sequence identity with SEQ ID NO: 10; and/or (ix) an amino acid sequence having at least 90% sequence identity with SEQ ID NO: 11.

In some embodiments, a peptide ligand from the present disclosure comprises: (i) an amino acid sequence having at least 95% sequence identity with SEQ ID NO: 3; (ii) an amino acid sequence having at least 95% sequence identity with SEQ ID NO: 4; (iii) an amino acid sequence having at least 95% sequence identity with SEQ ID NO: 5; (iv) an amino acid sequence having at least 95% sequence identity with SEQ ID NO: 6; (v) an amino acid sequence having at least 95% sequence identity with SEQ ID NO: 7; (vi) an amino acid sequence having at least 95% sequence identity with SEQ ID NO: 8; (vii) an amino acid sequence having at least 95% sequence identity with SEQ ID NO: 9; (viii) an amino acid sequence having at least 95% sequence identity with SEQ ID NO: 10; and/or (ix) an amino acid sequence having at least 95% sequence identity with SEQ ID NO: 11.

In some embodiments, a peptide ligand from the present disclosure comprises: (i) an amino acid sequence comprising SEQ ID NO: 3; (ii) an amino acid sequence comprising SEQ ID NO: 4; (iii) an amino acid sequence comprising SEQ ID NO: 5; (iv) an amino acid sequence comprising SEQ ID NO: 6; (v) an amino acid sequence comprising SEQ ID NO: 7; (vi) an amino acid sequence comprising SEQ ID NO: 8; (vii) an amino acid sequence comprising SEQ ID NO: 9; (viii) an amino acid sequence comprising SEQ ID NO: 10; and/or (ix) an amino acid sequence comprising SEQ ID NO: 11.

In some embodiments, the compositions of the present disclosure include: (i) at least two peptide ligands from SEQ ID NOs: 3-11; (ii) at least three peptide ligands from SEQ ID NOs: 3-11; (iii) at least four peptide ligands from SEQ ID NOs: 3-11; (iv) at least five peptide ligands from SEQ ID NOs: 3-11; (v) at least six peptide ligands from SEQ ID NOs: 3-11; (vi) at least seven peptide ligands from SEQ ID NOs: 3-11; (vii) at least eight peptide ligands from SEQ ID NOs: 3-11; or (viii) all nine peptide ligands of SEQ ID NOs: 3-11.

In some embodiments, a peptide ligand from the present disclosure comprises: (i) an amino acid sequence having at least 80% sequence identity with SEQ ID NO: 12; (ii) an amino acid sequence having at least 80% sequence identity with SEQ ID NO: 13; (iii) an amino acid sequence having at least 80% sequence identity with SEQ ID NO: 14; (iv) an amino acid sequence having at least 80% sequence identity with SEQ ID NO: 15; (v) an amino acid sequence having at least 80% sequence identity with SEQ ID NO: 16; (vi) an amino acid sequence having at least 80% sequence identity with SEQ ID NO: 17; (vii) an amino acid sequence having at least 80% sequence identity with SEQ ID NO: 18; (viii) an amino acid sequence having at least 80% sequence identity with SEQ ID NO: 19; and/or (ix) an amino acid sequence having at least 80% sequence identity with SEQ ID NO: 20.

In some embodiments, a peptide ligand from the present disclosure comprises: (i) an amino acid sequence having at least 85% sequence identity with SEQ ID NO: 12; (ii) an amino acid sequence having at least 85% sequence identity with SEQ ID NO: 13; (iii) an amino acid sequence having at least 85% sequence identity with SEQ ID NO: 14; (iv) an amino acid sequence having at least 85% sequence identity with SEQ ID NO: 15; (v) an amino acid sequence having at least 85% sequence identity with SEQ ID NO: 16; (vi) an amino acid sequence having at least 85% sequence identity with SEQ ID NO: 17; (vii) an amino acid sequence having at least 85% sequence identity with SEQ ID NO: 18; (viii) an amino acid sequence having at least 85% sequence identity with SEQ ID NO: 19; and/or (ix) an amino acid sequence having at least 85% sequence identity with SEQ ID NO: 20.

In some embodiments, a peptide ligand from the present disclosure comprises: (i) an amino acid sequence having at least 90% sequence identity with SEQ ID NO: 12; (ii) an amino acid sequence having at least 90% sequence identity with SEQ ID NO: 13; (iii) an amino acid sequence having at least 90% sequence identity with SEQ ID NO: 14; (iv) an amino acid sequence having at least 90% sequence identity with SEQ ID NO: 15; (v) an amino acid sequence having at least 90% sequence identity with SEQ ID NO: 16; (vi) an amino acid sequence having at least 90% sequence identity with SEQ ID NO: 17; (vii) an amino acid sequence having at least 90% sequence identity with SEQ ID NO: 18; (viii) an amino acid sequence having at least 90% sequence identity with SEQ ID NO: 19; and/or (ix) an amino acid sequence having at least 90% sequence identity with SEQ ID NO: 20.

In some embodiments, a peptide ligand from the present disclosure comprises: (i) an amino acid sequence having at least 95% sequence identity with SEQ ID NO: 12; (ii) an amino acid sequence having at least 95% sequence identity with SEQ ID NO: 13; (iii) an amino acid sequence having at least 95% sequence identity with SEQ ID NO: 14; (iv) an amino acid sequence having at least 95% sequence identity with SEQ ID NO: 15; (v) an amino acid sequence having at least 95% sequence identity with SEQ ID NO: 16; (vi) an amino acid sequence having at least 95% sequence identity with SEQ ID NO: 17; (vii) an amino acid sequence having at least 95% sequence identity with SEQ ID NO: 18; (viii) an amino acid sequence having at least 95% sequence identity with SEQ ID NO: 19; and/or (ix) an amino acid sequence having at least 95% sequence identity with SEQ ID NO: 20.

In some embodiments, a peptide ligand from the present disclosure comprises: (i) an amino acid sequence comprising SEQ ID NO: 12; (ii) an amino acid sequence comprising SEQ ID NO: 13; (iii) an amino acid sequence comprising SEQ ID NO: 14; (iv) an amino acid sequence comprising SEQ ID NO: 15; (v) an amino acid sequence comprising SEQ ID NO: 16; (vi) an amino acid sequence comprising SEQ ID NO: 17; (vii) an amino acid sequence comprising SEQ ID NO: 18; (viii) an amino acid sequence comprising SEQ ID NO: 19; and/or (ix) an amino acid sequence comprising SEQ ID NO: 20.

In some embodiments, the compositions of the present disclosure include: (i) at least two peptide ligands from SEQ ID NOs: 12-20; (ii) at least three peptide ligands from SEQ ID NOs: 12-20; (iii) at least four peptide ligands from SEQ ID NOs: 12-20; (iv) at least five peptide ligands from SEQ ID NOs: 12-20; (v) at least six peptide ligands from SEQ ID NOs: 12-20; (vi) at least seven peptide ligands from SEQ ID NOs: 12-20; (vii) at least eight peptide ligands from SEQ ID NOs: 12-20; or (viii) all nine peptide ligands of SEQ ID NOs: 12-20.

As would be recognized by one of ordinary skill in the art based on the present disclosure, the peptide ligands provided herein can be conjugated to a linker. In some embodiments, the linker can facilitate display of a peptide ligand onto a solid support, which allows for better capture of a target AAV, for example. In other embodiments, the peptide ligands provided herein are not conjugated to a linker, but can still bind to target AAVs and be purified from a cell culture fluid through other means. In some embodiments, the one or more peptide ligands comprise a linker on the C-terminus of the peptide. The C-terminus linker comprise a linker according to the following structure: Glyₙ or a [Gly-Ser-Gly]ₘ, wherein 6≥n≥1 and 3≥m≥1. The C-terminus linker can be any suitable linker including, but not limited to GSG and GGG.

In some embodiments, the cell culture fluid comprises a supernatant and/or a cellular lysate. In some embodiments, the cell culture fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof. In some embodiments, the cell culture fluid is derived from HEK cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof. In some embodiments, the cell culture fluid is derived from yeast cells. In some embodiments, the cell culture fluid is derived from a virus production cell line. In some embodiments, the virus production cell line is selected from the group consisting of MDCK-S, MDCK-A, Vero cells, LLC-MK2D, PER.C6, EB66, and AGE1.CR cells, Spodoptera frugiperda (Sf9) cells, and HeLa cells, or any derivatives or variants thereof.

In some embodiments, the cell culture fluid comprises a pH from about 3.0 to about 9.0. In some embodiments, the cell culture fluid comprises a pH from about 4.0 to about 9.0. In some embodiments, the cell culture fluid comprises a pH from about 5.0 to about 9.0. In some embodiments, the cell culture fluid comprises a pH from about 6.0 to about 9.0. In some embodiments, the cell culture fluid comprises a pH from about 7.0 to about 9.0. In some embodiments, the cell culture fluid comprises a pH from about 3.0 to about 8.0. In some embodiments, the cell culture fluid comprises a pH from about 3.0 to about 7.0. In some embodiments, the cell culture fluid comprises a pH from about 3.0 to about 6.0. In some embodiments, the cell culture fluid comprises a pH from about 4.0 to about 8.0. In some embodiments, the cell culture fluid comprises a pH from about 5.0 to about 7.0.

In accordance with the above embodiments, the at least one peptide ligand can comprise at least 6 peptide ligands, at least 7 peptide ligands, at least 8 peptide ligands, at least 9 peptide ligands, at least 10 peptide ligands, at least 11 peptide ligands, at least 12 peptide ligands, at least 13 peptide ligands, at least 14 peptide ligands, at least 15 peptide ligands, at least 16 peptide ligands, at least 17 peptide ligands, at least 18 peptide ligands, at least 19 peptide ligands, at least 20 peptide ligands, at least 21 peptide ligands, at least 22 peptide ligands, at least 23 peptide ligands, at least 24 peptide ligands, at least 25 peptide ligands, at least 26 peptide ligands, at least 27 peptide ligands, at least 28 peptide ligands, at least 29 peptide ligands, or at least 30 peptide ligands. In some embodiments, the one or more peptide ligands comprise different amino acid sequences.

b. Adsorbents

Further described herein are adsorbents comprising a composition as described above, where each peptide ligand of the composition is conjugated to a support. Supports may comprise, but are not limited to, particles, beads, plastic surfaces, resins, fibers, and/or membranes. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate, polyolefin, polyester, polysaccharide, iron oxide, silica, titania, and/or zirconia. In some embodiments, supports may include microparticles and/or nanoparticles. Each support may be made out of any suitable material including, but not limited to, synthetic or natural polymers, metals, and metal oxides. Some supports may be magnetic, such as a magnetic bead, microparticle and/or nanoparticle. Suitable synthetic polymers include, but are not limited to, polymethacrylate, polyethersulfone, and polyethyleneglycole. Suitable natural polymers include, but are not limited to, cellulose, agarose, and chitosan. Suitable metal oxides include, but are not limited to, iron oxide, silica, titania, and zirconia. Further described herein are adsorbents comprising a composition as described above conjugated to a support.

In some embodiments, the adsorbent comprises a single type of support made from a single type of support material, where all of the peptides in the composition are conjugated to supports formed of the single type of support material. In these embodiments, the composition may comprise one or more different types of peptides, each conjugated to the single type of support made from the single type of support material. In other embodiments, the adsorbent comprises a plurality of types of support. Each type of support may be made of the same type of support material or different types of support materials. In these embodiments, the composition may comprise one or more different types of peptides, as described further herein, each conjugated to a different type of support. In still other embodiments, the peptides of the composition can be conjugated to a soluble compound, for example stimuli-responsive polymer chains to remove AAVs by affinity precipitation.

c. Methods

As described further herein, the present disclosure also provides improved methods for purifying an AAV from a biological fluid comprising one or more product- and/or process-related impurities or contaminants, as compared to currently used methods. In some embodiments, the method includes contacting a composition comprising any of the peptide ligands described herein, or an adsorbent comprising any of the peptides described herein, with a biological fluid comprising the AAV, wherein the at least one peptide ligand binds the AAV. In accordance with these embodiments, the method includes eluting the AAV from the peptide ligand, thereby purifying the AAV. The methods of the present disclosure can further comprise washing the composition or adsorbent to remove one or more product- and/or process-related impurities or contaminants from the AAVs bound to the peptide ligands. In some embodiments, the method can be performed under any binding conditions suitable for use with the composition or adsorbent, including both static binding conditions and dynamic binding conditions.

As described further herein, the peptide ligands of the present disclosure exhibit surprising and unexpected advantages over the compositions and methods currently available to purify AAVs. For example, currently available ligands that are used to purify AAVs require harsh elution conditions (e.g., pH<3.0), which damages the AAV products being eluted. In contrast, the peptide ligands of the present disclosure release bound AAVs under much gentler conditions, which do not damage the AAV product. In accordance with this, embodiments of the present disclosure include at least one peptide ligand from (a) and/or (b) that exhibits a disassociation constant (K_{D}) higher than or equal to about 10⁻⁴ M at a pH ranging from about 6.0 to 7.5.

The binding affinity of the compositions and/or adsorbent for the AAVs, as compared to one or more product- and/or process-related impurities or contaminants, can be altered by changes in the following: properties and concentration of the one or more product- and/or process-related impurities or contaminants; the properties and concentration of the host cell proteins; the composition, concentration, and pH of the mixture; and/or the loading conditions and residence time of the contacting and washing steps. Any of these variables can be changed to variables which are suitable according to the methods of the present disclosure and result in increased or decreased binding affinity as required for the present disclosure.

In some embodiments, the contacting step can comprise a low pH buffer of between pH 5-9. In some embodiments, the contacting step can comprise a low pH buffer of between pH 5-8. In some embodiments, the contacting step can comprise a low pH buffer of between pH 5-7. In some embodiments, the contacting step can comprise a low pH buffer of between pH 6-9. In some embodiments, the contacting step can comprise a low pH buffer of between pH 6-8. In some embodiments, the contacting step can comprise a low pH buffer of between pH 6-7. In some embodiments, the contacting step can comprise a low pH buffer of between pH 7-9. In some embodiments, the contacting step can comprise a low pH buffer of between pH 7-8.

In some embodiments, the elution is performed at pH from about 5.0 to about 8.0. In some embodiments, the elution is performed at pH from about 5.0 to about 7.5. In some embodiments, the elution is performed at pH from about 5.0 to about 7.0. In some embodiments, the elution is performed at pH from about 5.0 to about 6.5. In some embodiments, the elution is performed at pH from about 5.0 to about 6.0. In some embodiments, the elution is performed at pH from about 5.0 to about 5.5. In some embodiments, the elution is performed at pH from about 5.5 to about 8.0. In some embodiments, the elution is performed at pH from about 6.0 to about 8.0. In some embodiments, the elution is performed at pH from about 6.5 to about 8.0. In some embodiments, the elution is performed at pH from about 7.0 to about 8.0. In some embodiments, the elution is performed at pH from about 7.5 to about 8.0. In some embodiments, the elution is performed at pH from about 6.0 to about 7.0. In some embodiments, the elution is performed at pH from about 5.5 to about 7.5.

In some embodiments, the methods of the present disclosure result in at least a 50% yield for the AAV (e.g., as compared to methods in which the peptide ligands of the present disclosure are not used). In some embodiments, the methods of the present disclosure result in at least a 60% yield for the AAV. In some embodiments, the methods of the present disclosure result in at least a 70% yield for the AAV. In some embodiments, the methods of the present disclosure result in at least an 80% yield for the AAV. In some embodiments, the methods of the present disclosure result in at least a 90% yield for the AAV.

In some embodiments, the methods of the present disclosure produce at least an 80-fold reduction in host cell proteins when purifying an AAV (e.g., as compared to methods in which the peptide ligands of the present disclosure are not used). In some embodiments, the methods of the present disclosure produce at least a 100-fold reduction in host cell proteins. In some embodiments, the methods of the present disclosure produce at least a 150-fold reduction in host cell proteins. In some embodiments, the methods of the present disclosure produce at least a 200-fold reduction in host cell proteins. In some embodiments, the methods of the present disclosure produce at least a 250-fold reduction in host cell proteins. In some embodiments, the methods of the present disclosure produce at least a 300-fold reduction in host cell proteins. In some embodiments, the methods of the present disclosure produce at least a 350-fold reduction in host cell proteins. In some embodiments, the methods of the present disclosure produce at least a 400-fold reduction in host cell proteins.

Embodiments of the present disclosure also include an adeno-associated virus (AAV) purified using any of the methods described herein. In some embodiments, the AAV exhibits at least 50% transduction activity. In some embodiments, the AAV exhibits at least 60% transduction activity. In some embodiments, the AAV exhibits at least 70% transduction activity. In some embodiments, the AAV exhibits at least 80% transduction activity. In some embodiments, the AAV exhibits at least 90% transduction activity.

### 3. Examples

Viral vectors—and, chiefly among them, adeno-associated viruses (AAVs) —are poised to become the focus of next-generation treatment of rare cardiovascular, muscular, neurological, and ophthalmological diseases. Following the approval of LUXTURNA® (AAV9) for the treatment of retinal dystrophy (2019) and ZOLGENSMA® (AAV2) for treating pediatric patients with spinal muscular atrophy, late in 2022 The U.S. FDA approved CSL's HEMGENIX®, an AAV5-based gene therapy for hemophilia B. With the expected uptick in regulatory approvals and the exponential growth of the clinical trials adsorbents on viral vector-based gene therapies poses a defined need for affordable tools for manufacturing AAV-based therapies, which currently carry price tags over $2.5M. In this context, a key role will be played by purification technology and particularly by affinity adsorbents for universal AAV purification. The adsorbents currently on the market have drawn substantially—both in the morphology of the beads and the design of the ligands—from the Protein A adsorbents employed in antibody purification, and are therefore poorly suited for AAV purification. Responding to these challenges, embodiments of the present disclosure provide the development of the first set of serotype-agnostic AAV-targeting peptide ligands. The use of synthetic peptide ligands is particularly apt to the purification of viral vectors, since it (i) limits the cost of the adsorbents to below $8K per liter (note: this value includes the costs of the base resin, the purified peptide, and the ligand conjugation to produce 100 liters of adsorbent) against the $24-60K per liter of commercial resins; (ii) enables promiscuous targeting of conserved epitopes on the surface of the AAV capsids while ensuring sufficient selectivity to afford a significant reduction of process-related impurities; and (iii) features a moderate binding strength, thus enabling efficient elution of the bound capsids under near-physiological conditions, which are much milder than those requires for commercial affinity resins and afford an AAV product with superior transduction activity. The combination of universal serotype targeting, high binding capacity, excellent purification activity, and safeguarding the capsid's transduction activity at a fraction of the cost of commercial adsorbents make these peptide-based adsorbents valuable tools for large scale applications supporting clinical efforts.

The accompanying Examples are offered as illustrative as a partial scope and particular embodiments of the disclosure and are not meant to be limiting of the scope of the disclosure.

### Example 1

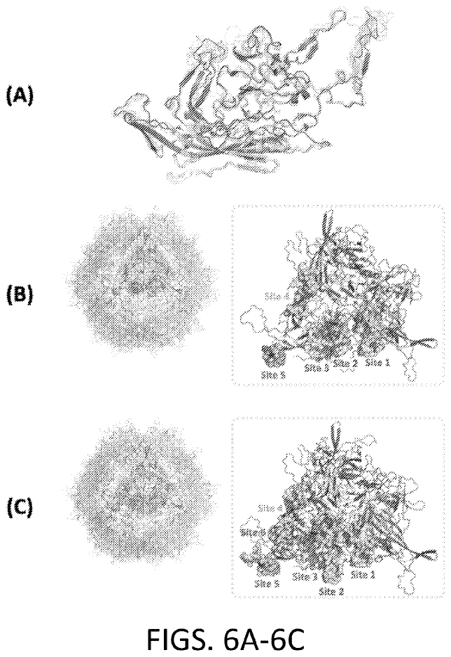

Selection of AAV-targeting ligands via rational design and screening of combinatorial peptide libraries. The biomolecular features that differentiate the various AAV serotypes—namely, the amino acid sequences of the virion proteins VP1, VP2, and VP3, and their unique arrangement within the capsid—also determine their behavior in terms of tissue tropism, transduction efficiency, and patient safety. These domains are displayed on the protrusions found on the fivefold cylinder and on the lines drawn between two contiguous threefold axes, and the twofold and fivefold axes. As active sites, however, these domains are not suitable binding targets, since the association and dissociation with affinity ligands may cause structural and biochemical alterations leading to unwanted loss of tissue tropisms and transduction efficiency. Conversely, highly conserved regions (CRs) are found in all serotypes' capsids, including a core eight-stranded β-barrel motif (BB-BI) and a α-helix (αA) on the convex side of the VPs (FIG. 6A), that are not implicated in receptor binding, transduction, and antigenic specificity. These represent ideal target regions to universal AAV-binding peptides serving as ligands for serotype-independent purification of AAVs from recombinant fluids. To gather molecular-level insight in the molecular landscape of the AAV surface, an in silico “druggability” study of the CRs was performed using PockDrug and identified 5 candidate sites whose morphology and physicochemical properties are suitable for docking peptide ligands (FIGS. 6b-6C, and Table 1).

TABLE 1
Structural and biophysical properties of target sites on
AAV Capsids. Properties of the putative binding sites
identified via SiteMap on the homologous, solvent accessible
peptide segments displayed on the convex side of the of
VP1-VP2-VP3 cluster of AAV2 (PDB ID: 6IH9) and AAV9 (7WJX);
the binding sites are labeled in FIG. 6.

| | | | | | | |
|---|---|---|---|---|---|---|
| Site | SAS (Å²) | Volume (Å³) | Ip | Gravy | Polarity | Score |

AAV2

| | | | | | | |
|---|---|---|---|---|---|---|
| 1 | 265.7 | 1953.9 | 8.23 | −0.605 | 0.583 | 0.892 |
| 2 | 290.3 | 2832.5 | 7.85 | −0.543 | 0.645 | 0.890 |
| 3 | 277.0 | 2079.8 | 7.48 | −0.470 | 0.470 | 0.833 |
| 4 | 295.7 | 2121.7 | 8.47 | −0.403 | 0.663 | 0.907 |
| 5 | 154.9 | 869.8 | 4.63 | −0.335 | 0.585 | 0.743 |

AAV9

| | | | | | | |
|---|---|---|---|---|---|---|
| 1 | 214.9 | 1917.4 | 8.72 | −0.668 | 0.564 | 0.862 |
| 2 | 348.4 | 2703.6 | 7.64 | −0.612 | 0.636 | 0.937 |
| 3 | 322.6 | 1907.2 | 7.27 | −0.628 | 0.434 | 0.812 |
| 4 | 270.1 | 1689.1 | 8.27 | −0.610 | 0.573 | 0.927 |
| 5 | 176.9 | 969.8 | 4.55 | −0.393 | 0.537 | 0.723 |
| 6 | 182.2 | 951.2 | 6.72 | −0.487 | 0.343 | 0.862 |

The physicochemical properties of the selected sites guided the design of a peptide library for the selection of candidate ligands: (i) a chain length of 6 or 8 monomers was chosen based on the average size of the pockets (Vander Waals volume~950-1150 Å³; projection area~150-250 Å²), which provides an ideal balance—based on prior knowledge—between the expected biorecognition activity and manufacturing cost; (ii) the combinatorial positions in the library were randomized with alanine, asparagine, glutamic acid, histidine, isoleucine, lysine, phenylalanine, serine, and tryptophan, which were adopted as the amino acids capable of forming a network of diverse non-covalent interactions with the selected binding pockets; and (iii) a Gly-Ser-Gly (GSG) tripeptide spacer was utilized to link the combinatorial segment of the library to the resin to improve peptide display, thus promoting the outcome of library screening and subsequent Edman sequencing. The peptide libraries were synthesized following the “split-couple-and-recombine” technique on ChemMatrix beads—porous, hydrophilic, translucent particles that have proven an excellent substrate for the synthesis and selection of peptide ligands.

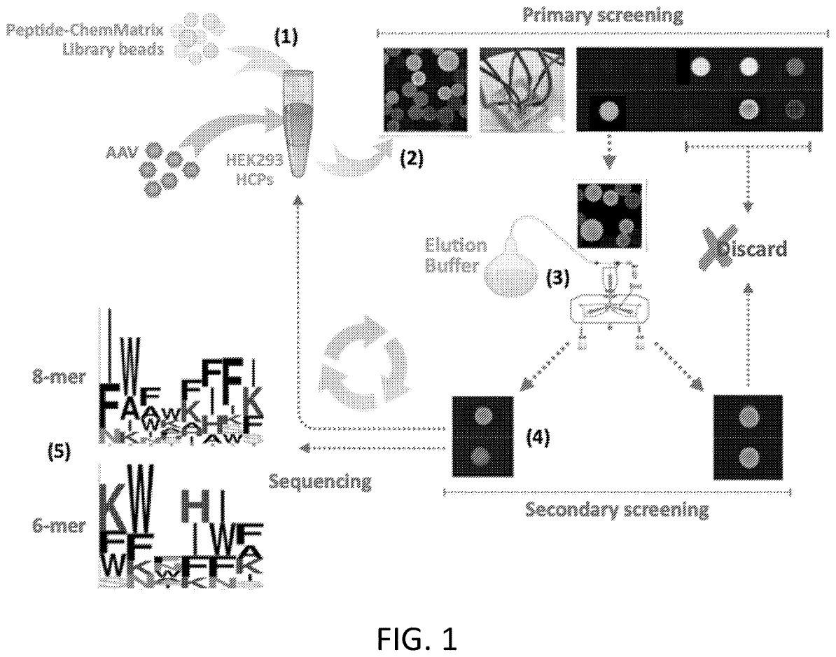

The library was incubated with a model feedstock containing red-fluorescently labeled AAV2 and green-labeled HEK 293 host cell proteins (HCPs), and screened using a bead sorting device developed for the rapid selection of peptide ligands: AAV2 was utilized as model target for being the most studied and utilized of the currently known human and nonhuman primate AAV serotypes; the formulation of the feedstock—namely AAV2 at 5·10¹¹ vp/mL and HEK 293 HCPs at 0.5 mg/mL—mimics industrial cell culture lysates and was adopted to identify peptide ligands capable of isolating AAV from complex sources in bind-and-elute mode. The device comprises a microfluidic chamber, where each bead is imaged using a multiple wavelength fluorescence microscope, and is controlled by a software performing real-time monitoring, image processing, and selection of the beads (FIG. 1A). Beads with high binding strength (i.e., ratio of the bead's vs. standard red fluorescence intensity >0.9) and selectivity (i.e., red vs. green intensity ratio >100) are retained in the device, while all other beads are discarded. Each retained bead is exposed to a flow of elution buffer, namely 1 M MgCl₂ in 20 mM Bis-Tris buffer at pH 6.0, whose composition and pH was adopted to ensure the selection of peptide ligands that enable efficient AAV release under gentle conditions. Accordingly, only the beads displaying effective AAV elution (i.e., ratio of bead's pre-vs. post-elution red fluorescence intensity >10) were selected and analyzed via Edman degradation to sequence the peptide carried thereon. The resulting 6-mer and 8-mer sequences are listed in Table 2, while the homology analysis is in FIG. 1B.

TABLE 2
Sequences and biophysical properties of selected 6-mer and 8-mer AAV-
binding peptide ligands. The values of isoelectric point (pI),
polarity (Grantham scale), and Grand Average Hydropathy index (GRAVY)
were calculated based on the amino acid sequence and
assuming an amidated C-terminus to represent the conjugation
of the peptide to the chromatographic resin.

| Sequence | pI | Polarity | GRAVY |
|---|---|---|---|
| FFNFFK | 10.13 | 5.65 | -1.13 |
| (SEQ ID NO: 3) | | | |
| SWFIIF | 9.01 | 5.47 | -1.95 |
| (SEQ ID NO: 4) | | | |
| KWFIIF | 11.13 | 5.88 | -1.50 |
| (SEQ ID NO: 5) | | | |
| FNEFFI | 6.99 | 5.22 | -1.02 |
| (SEQ ID NO: 6) | | | |
| WKAHNK | 11.66 | 5.03 | 0.3 |
| (SEQ ID NO: 7) | | | |
| WWIKIS | 11.03 | 5.64 | -1.18 |
| (SEQ ID NO: 8) | | | |
| KFNHWF | 10.93 | 5.78 | -0.95 |
| (SEQ ID NO: 9) | | | |
| KWWIWA | 11.23 | 6.18 | -1.58 |
| (SEQ ID NO: 10) | | | |
| KWWHWA | 10.83 | 6.24 | -1.37 |
| (SEQ ID NO: 11) | | | |
| FWNWHHFK | 11.9 | 7.95 | -1.20 |
| (SEQ ID NO: 12) | | | |
| IWAWFHFF | 10.9 | 7.86 | -2.14 |
| (SEQ ID NO: 13) | | | |
| FWWAAFFK | 10.2 | 7.55 | -1.54 |
| (SEQ ID NO: 14) | | | |
| IWWHIAKF | 10.9 | 7.44 | -1.36 |
| (SEQ ID NO: 15) | | | |
| IKIFFFFS | 11.8 | 7.04 | -1.29 |
| (SEQ ID NO: 16) | | | |
| NWAWFIWK | 11.8 | 7.81 | -1.48 |
| (SEQ ID NO: 17) | | | |
| FIFSKFFI | 11.5 | 7.03 | -1.29 |
| (SEQ ID NO: 18) | | | |
| IAFKKISI | 12.1 | 6.02 | -0.26 |
| (SEQ ID NO: 19) | | | |
| IAFKKIII | 11.7 | 6.33 | -0.53 |
| (SEQ ID NO: 20) | | | |

### Example 2

Evaluation of AAV binding and release by peptide-functionalized chromatographic resins in non-competitive mode. Selected sequences IWWHIAKF, FWNWHHFK, FWWAAFFK, IAFKKISI, IKIFFFFS, GYISRHPG, KFNHWF, WKAHNK, KWWIWA, WWIKIS, FFNFFK, and FNHFFI were conjugated to Toyopearl AF-Amino-650M resin and evaluated for AAV binding and elution in non-competitive mode to draw an initial ranking of the candidate ligands; commercial affinity adsorbents POROS™ CaptureSelect™ AAVX Affinity and AVB Sepharose HP resin were utilized as reference standards. Since different serotypes display specific tissue tropisms, evaluating the ability of an affinity resin to target different serotypes is the first step in demonstrating its potential in downstream bioprocessing of AAVs. In this context, AAV2 and AAV9 were adopted as model serotypes: AAV2 is the most widely studied serotype to date and is the one for which the majority of values of binding capacity of affinity adsorbents are reported in the technical literature; AAV9 has received significant attention—clinically, for its ability to bypass the blood-brain barrier (BBB) and, in the context of biomanufacturing, for being a secreted vector whose purification is challenging (note: affinity resins marketed as universal AAV binders often struggle to capture AAV9, and dedicated adsorbents for AAV9 purification have been developed). To evaluate the peptide-based resins under industrially relevant conditions, pure AAV2 and pure AAV9 at ~5·10¹¹ vp/mL in 10 mM Bis-Tris buffer at pH 7.0 were utilized as feedstocks, and loaded at a ratio of ~10¹³ vp per mL of resin, which is expected to be the average binding capacity of the resins.

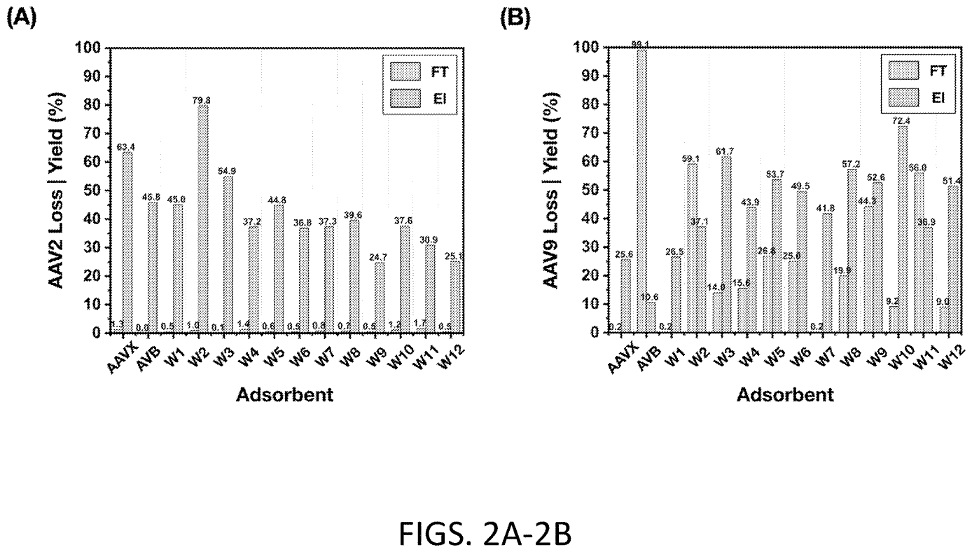

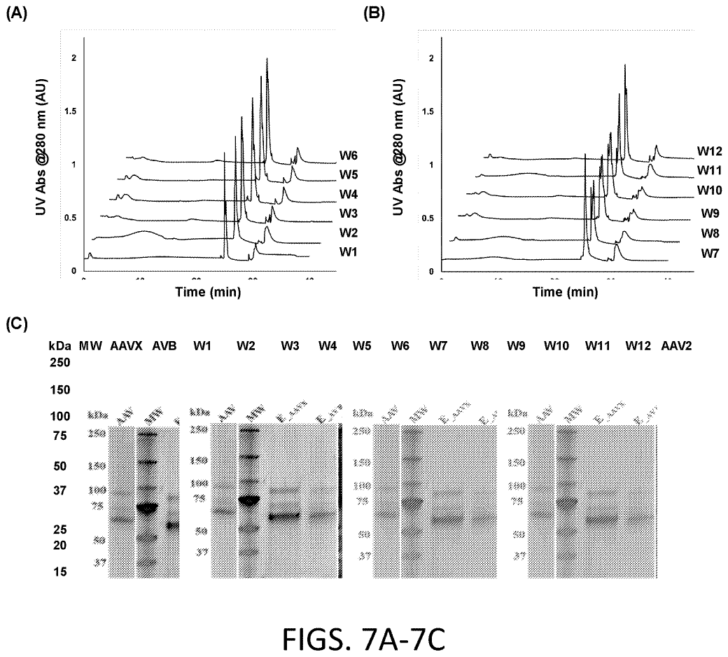

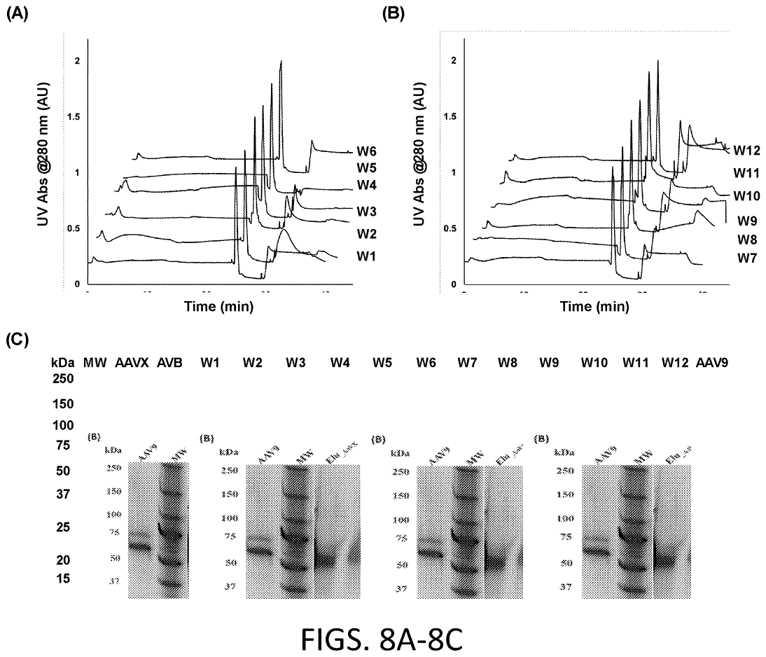

The bound AAV vectors were recovered from the peptide-Toyopearl resins under the same mild elution conditions adopted in library screening—namely, 1 M MgCl₂ in 20 mM Bis-Tris buffer at pH 6.0—while strong acidic elution (i.e., 200 mM MgCl₂ in 200 mM citrate buffer at pH 2.2 and PBS at pH 2.0, respectively, as recommended by the manufacturers) were implemented for the AAVX and AVB resins to ensure the most stringent performance evaluation of the selected ligands. The chromatograms obtained with pure AAV2 and AAV9 and the electrophoretic analysis of the collected fractions are respectively reported in FIG. 7 and FIG. 8, while the values of yield are reported in FIG. 2.

As shown in FIG. 2, all peptide resins bound AAV2 efficiently (<1.7% loss). These results corroborate the library design criteria inspired by the druggability study of AAV capsid proteins and are in line with the in-silico evaluation of AAV:peptide binding reported below. Specifically, the values of AAV2 yield provided by KFNHWFG (45%), WKAHNKG (79.8%), IWWHIAKFG (54.9%), and FWWAAFFKG (44.8%) were comparable to those granted by AAVX POROS™ (63.4%) and AVB Sepharose (45.8%) resins. Similar values of yield were obtained with AAV9: noteworthily, IWWHIAKFG (61.7%), FWNWHHFKG (43.9%), FWWAAFFKG (53.7%), KWWIWAG (57.2%), and FFNFFKG (72.4%) significantly outperformed AAVX POROS™ (25.6%) and AVB Sepharose (10.6%) resins. Notably, while the values of yield of both serotypes were comparable across the various resins, the values of product loss in the flow-through and wash were substantially higher for AAV9 than AAV2. On the other hand, the values of yield and binding strength calculated in silico (K_{D,in-silico}, see Table 3) indicate that product loss is not due to lack of affinity by the peptides for AAV9. The electrophoretic analysis of eluted AAV2 (FIG. 7) and AAV9 (FIG. 8) clearly show the presence of all three capsid proteins VP1 (~87 kDa), VP2 (~73 kDa), and VP3 (~62 kDa) in the correct ~1:1:10 ratio, based on the densitometric analysis of the gels, corroborating the interpretative hypothesis that only fully formed capsids are captured by the peptide ligands.

Overall, the performance of the peptide ligands is rather remarkable—with respect to their protein counterparts—when one considers the difference in elution conditions (pH 6 vs. pH 2). At low pH, in fact, AAVs can undergo conformational alterations that cause capsid aggregation or loss of integrity; externalization of the VP1 phospholipase A2 (PLA₂) domain, which triggers capsid uncoating and release of the genetic payload; and biochemical alterations (e.g., hydrolysis, deamidation, or oxidation of the VPs) that impair capsid docking to the cellular receptor (AAVR), and thus host entry and trafficking. The current approach to minimizing these instances relies on immediate pH neutralization of the elution stream from POROS™ AAVX and AVB Sepharose resins. Conversely, the unique approach enabled by the peptide presented in this study aims to preserve the transduction activity of the AAV products by achieving efficient elution under near-physiological conditions.

### Example 3

In silico investigation of AAV:peptide binding. The results presented above suggest that the identified peptide sequences are capable of binding not only the AAV2 capsids employed in the library screening, but also AAV9. Notably, these two serotypes belong to different antigenic clades, respectively B and F, and feature rather different sequences (VP1/VP3 amino acid identity ~81%) and structures (structural identity ~94%).

To evaluate the ability of the selected peptides to bind multiple AAV serotypes, the binding of three 6-mer (KFNHWFG, FFNFFKG, and FNHFFIG) and three 8-mer (IWWHIAKFG, FWNWHHFKG, and FWWAAFFKG) peptides were modeled to the crystal structures of AAV2, AAV6, AAV8, and AAV9 capsids via molecular docking and molecular dynamics (MD). The secondary structure of the peptides, obtained via MD simulations in explicit water, were docked against a spherical cap representing the AAV capsids in HADDOCK v. 2.4. The homology spherical cap structures were obtained by collating published structures, namely PDB IDs 6U0V, 6IH9, 5IPI, and 6IHB for AAV2; 3SHM, 5EGC, 4V86, 3OAH for AAV6; 6V10, 2QA0, 3RAA, 6U2V, 6PWA for AAV8; and 3UX1, 7MT0, 7WJW, and 7WJX for AAV9. An initial round of “blind” docking was performed to evaluate—in an unbiased fashion—the ability of the selected sequences to target the homologous binding sites identified in the initial “druggability” study (FIG. 6B). Additionally, to mimic the orientational constraint imposed upon the peptides by their conjugation onto the surface of the chromatographic resin, the -GSG tripeptide was appended on the C-terminal end of the peptides not to bind AAV.

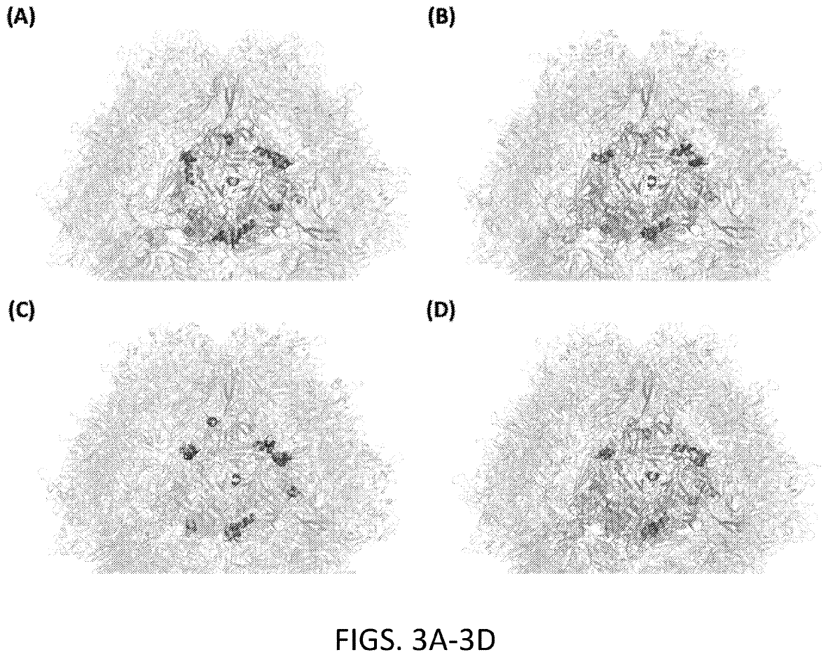

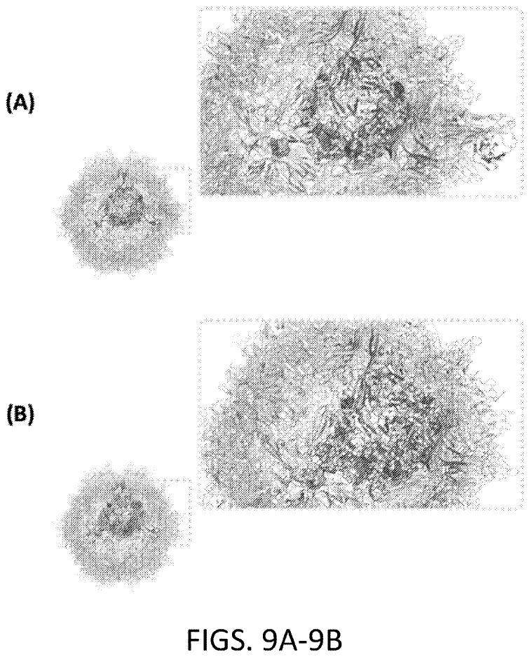

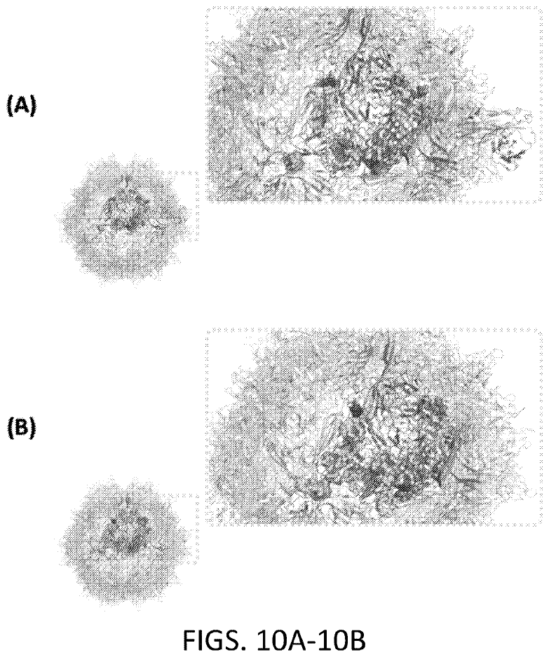

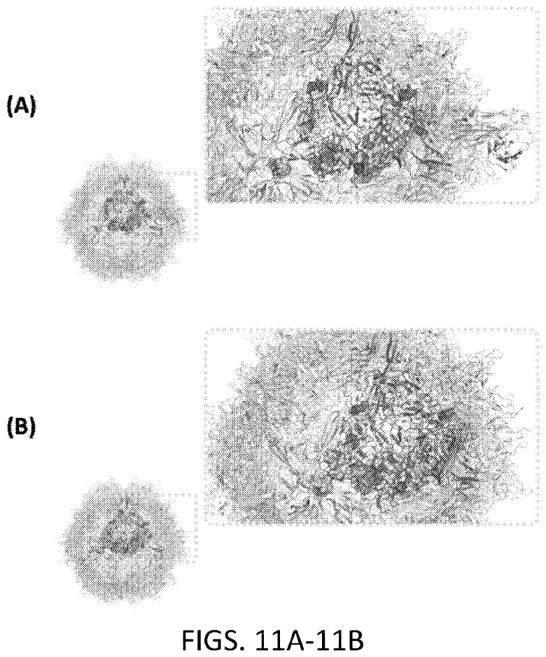

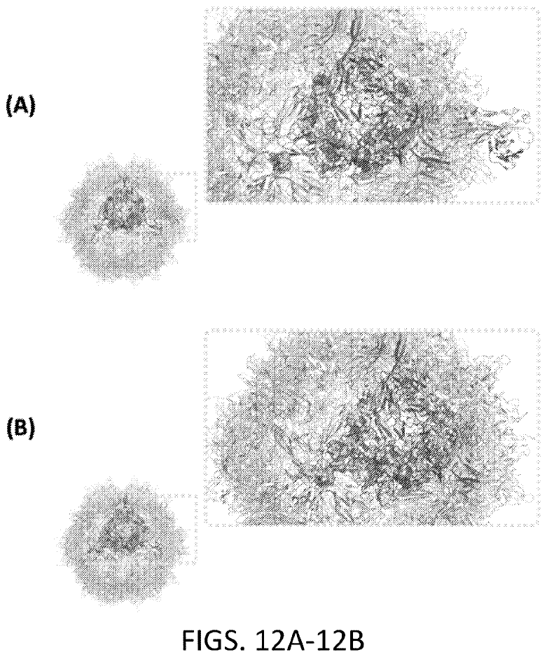

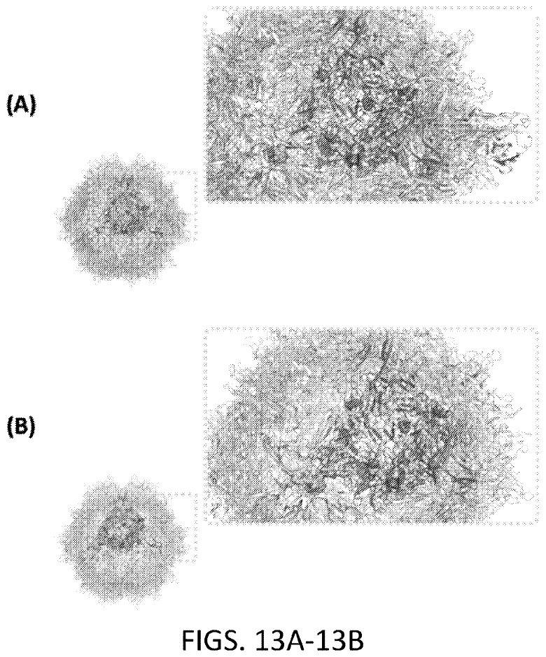

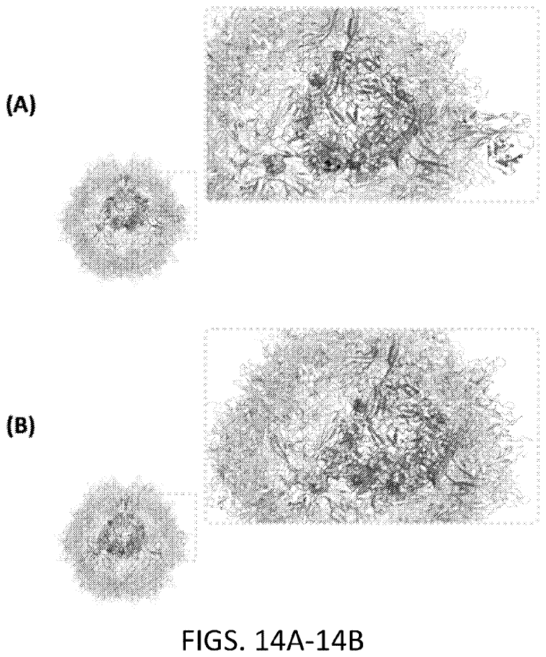

The resulting AAV:peptide complexes were refined via MD simulations (150 ns) in explicit solvent to obtain values of Gibbs free energy of binding (ΔG_{b}), which were used to identify putative binding sites (|ΔG_{b}|>6.5 kcal/mol). Notably, all binding poses identified on AAV2 and AAV9 coincided with the binding sites identified in the “druggability” study. Accordingly, a second round of peptide docking was performed on these sites upon conditioning the homology structures of the capsids to both pH 7.4 and 6.0, and the docked structures were refined via extended MD simulations (500 ns) to obtain accurate values of binding energy. Representative complexes formed by the selected peptides with AAV2, AAV6, AAV8, and AAV9 capsids at pH 7.4 are shown in FIG. 3, while the values of AGE and the corresponding values of dissociation constant (K_{D,in silico}) at pH 7.4 and pH 6 are listed in Table 3; finally, detailed results of peptide docking for AAV2 and AAV9 are reported in FIGS. 9-14.

Confirming the criteria of library design and the results of dynamic binding in FIG. 2, the in silico results demonstrate the ability of the selected peptides to target AAVs in a serotype-agnostic manner. Specifically, the peptides consistently conserved regions located at the interface among different VPs, which is critical in order for AAV-binding ligands to target not only multiple serotypes but also capsids of the same serotype, given the stochastic arrangement of the VPs within a capsid. Among the docked peptides, KFNHWFG and IWWHIAKFG in particular targeted homologous binding sites located at the VP1-VP2, VP1-VP3, VP2-VP3, and VP3-VP3 interfaces, while FWNWHHFKG and FNHFFIG only targeted the VP1-VP3, VP2-VP3, and VP3-VP3 interfaces. Analogous behavior is found among anti-AAV antibodies, especially those utilized in analytical and diagnostic kits. As portrayed in FIG. 3, and in more detail in FIGS. 9-14, the pose of each peptide on homologous target sites located at different interfaces varies slightly due to subtle variations in the mutual orientation of the interlocking VPs. Because this translates in minor differences in the peptide:capsid binding energy, the values of K_{D,in silico} reported in Table 3 were derived from the average of the AGE of the various poses weighted by the frequency of the interfaces. Furthermore, peptides FNHFFIG, KFNHWFG, and IWWHIAKFG were also found to target conserved druggable domains displayed on VP3, and hence on VP1 and VP2, although they did not overlap with the binding sites of the AAV receptor (AAVR, in cyan in FIGS. 9-14).

TABLE 3
Values of dissociation constant (K_{D,in silico})
of the complexes formed by peptides
KENHWFG, FFNFFKG, FNHFFIG, IWWHIAKFG,
FWNWHHFKG, and FWWAAFFKG with the capsids
of AAV2, AAV6, AAV8, and AAV9 obtained
via molecular docking and dynamics
simulations. The values of K_{D,in silico}
were derived from the average of the
ΔG_{b} of the various VP: peptide complexes
weighted by the frequency of the interfaces,
namely 3.4% for VP1-VP2, 6.9% for
VP1-VP3 and VP2-VP3, and 82.8% for VP3-VP3.

| | AAV2: peptide | | AAV6: peptide | | AAV8: peptide | | AAV9: peptide | |
|---|---|---|---|---|---|---|---|---|
| | K_{D,in silico} (M) | | K_{D,in silico} (M) | | K_{D,in silico} (M) | | K_{D,in silico} (M) | |
| Sequence | pH 6.0 | pH 7.4 | pH 6.0 | PH 7.4 | pH 6.0 | PH 7.4 | pH 6.0 | pH 7.4 |
| KFNHWFG | 1.2·10^{—4} | 1.3·10 ⁶ | 7.5·10 ⁴ | 2.0·10 ⁶ | 1.9·10 ⁴ | 2.6·10 ⁶ | 6.1·10 ⁵ | 4.6·10 ⁷ |
| FFNFFKG | 5.4·10 ⁴ | 1.1·10 ⁶ | 7.2·10 ⁴ | 3.7·10 ⁶ | 7.2·10 ⁴ | 1.0·10 ⁵ | 1.0·10 ³ | 1.3·10 ⁶ |
| FNHFFIG | 1.6·10 ⁴ | 9.3·10 ⁷ | 4.2·10 ⁴ | 5.2·10 ⁶ | 6.6·10 ⁶ | 1.3·10 ⁷ | 2.4·10 ³ | 3.7·10 ⁵ |
| IWWHIAKFG | 5.1·10 ³ | 2.4·10 ⁶ | 1.1·10 ³ | 2.7·10 ⁶ | 7.2·10 ⁴ | 2.9·10 ⁶ | 4.8·10 ³ | 1.1·10 ⁵ |
| FWNWHHFKG | 5.4·10 ⁴ | 1.9·10 ⁶ | 7.2·10 ⁴ | 2.4·10 ⁶ | 2.7·10 ⁴ | 5.1·10 ⁵ | 3.7·10 ³ | 1.3·10 ⁵ |
| FWWAAFFKG | 1.3·10 ³ | 1.7·10 ⁶ | 6.4·10 ⁴ | 2.3·10 ⁶ | 1.7·10 ⁴ | 8.1·10 ⁶ | 5.0·10 ³ | 2.5·10 ⁵ |

Notably, the binding strength of the various site:peptide complexes was found to be rather weak compared to the values of the AAV:antibody counterparts (K_{D,in silico}~10⁻⁹ M). Notably, the 6-mer sequences KFNHWFG, FFNFFKG, and FNHFFIG consistently display a higher affinity, with values of K_{D,in silico} across the four serotypes fluctuating between 10⁻⁶ and 10⁻⁷ M, whereas 8-mers IWWHIAKFG, FWNWHHFKG, and FWWAAFFKG ranked as weaker binders, with K_{D,in silico}~10⁻⁵, 10⁻⁶ M. The trajectories of the molecular dynamic simulations indeed showed that the 6-mer peptides outperformed 8-mer peptides in accessing the druggable pockets located in the valleys located between the capsid's threefold and twofold axes. Furthermore, the analysis of pairwise interactions between the residues displayed by the peptide ligands and key amino acids in the target sites of VP3 demonstrate the formation of a dense network of side chain-side chain and side chain-backbone hydrogen bonds as well as π-π interactions; these account respectively for 56-68% and 11-16% of the VP:peptide binding energy; notably, fewer-than-expected coulombic and hydrophobic interactions were recorded, which provided lesser contributions, respectively 8-14% and 7-12%, to the binding energy.

Moderate binding strength is welcome in the context of affinity chromatography of labile therapeutics. For AAV purification in particular, weak VP:peptide interactions are conductive to easier elution and reduce the risk of capsid adsorption resulting in the denaturation of the protrusions that determine tissue tropism and gene transduction to the target cells. At the same time, the peptide density on the surface of the resin is sufficient to achieve multi-site interactions that grant high binding capacity and efficient product capture despite the low titer of capsids in the feedstock. Based on the values of peptide density on the resin (~0.12-0.15 mmol per gram) and the resin's specific surface (~30 m²/g), and projection area of each asymmetric unit on the icosahedral capsid (~81 nm²)¹, approximately 30 peptides are displayed on the area of the resin that is impacted by a single capsid. Comparing this number with the arrangement of the peptide binding poses on the triangular unit shown in FIG. 3 shows the likelihood of forming 3-5 VP:peptide interactions per bound capsid. This ultimately suggests that AAV capture by the peptide-functionalized resin is governed by a multi-site binding mechanism, where the μM-level affinity of single peptides are synergized into nM-level avidity, on par with AAV:antibody binding. It was further speculated that molecular-level non-idealities in the display of peptides or the posture of the capsid on the resin surface curb the avidity-driven capture, preventing irreversible adsorption of most of the loaded capsids; at the same time, where ideal binding arrangements occur, the mild elution conditions may fail to release the adsorbed capsids.

Following the description of the adsorption step, the in silico results also help elucidate AAV elution under mild conditions by offering a mechanism of capsid dissociation from the resin-bound peptides. Specifically, the molecular dynamic simulations show that the residues located at the periphery and in the immediate surrounding of the binding sites participate in a cyclical opening/closing conformational change. Decreasing the pH from 7.4 to 6.0 lowers the electrostatic charge of the binding sites by neutralizing the histidine residues (pKₐ~6.0), the majority of which are neighbored by other cationic (i.e., lysine and arginine) or anionic (i.e., aspartate and glutamate); the analysis of primary sequences reported on PDB for AAV2 (6U0V), AAV6 (3SHM), AAV8 (6V10), and AAV9 (3UX1), indicates that 64% of histidine residues are either neighbored by a charged residue either immediately or with one interposing amino acid. As the local network of electrostatic bonds decrease upon acidification, the conformational flexibility of the binding sites increases, and so does the pulsatile behavior of the binding sites. As observed in prior work, oscillation and distortion of the pairwise interactions result in lower VP:peptide binding strength: specifically, the values of |ΔG_{b}| averaged over the last 100 ns of MD simulations decrease between pH 7.4 and 6.0 of ~3.5 kcal/mol for AAV2, ~3.3 kcal/mol for AAV6, ~2.2 kcal/mol for AAV8, and ~3.2 kcal/mol for AAV9. This ultimately translates in 50-200-fold variations in binding strength (K_{D,in silico}), which is consistent with the high values of recovery obtained experimentally. Furthermore, the addition of Mg²⁺—a kosmotropic cation—in the elution buffer promotes a salting-in effect, leading to the hydration of the binding sites and their dissociation from the ligands. This combination of conformational pulsing and salting-in of the binding sites represents a powerful elution trigger and supports the high values of AAV yield granted by the peptide ligands.

Collectively, the in silico results corroborate the experimental observations that the selected sequences bind AAVs in a serotype-agnostic manner and afford efficient elution of bound capsids under mild conditions.

### Example 4

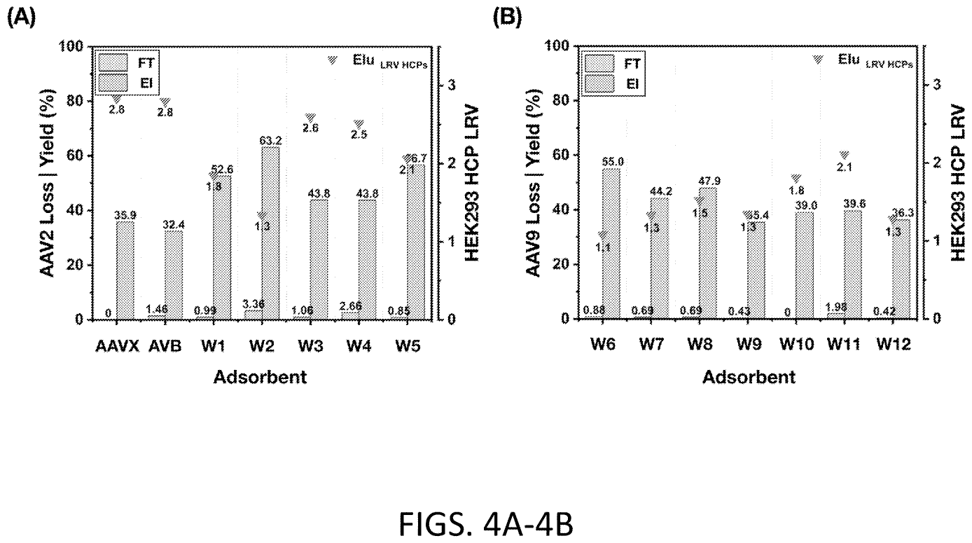

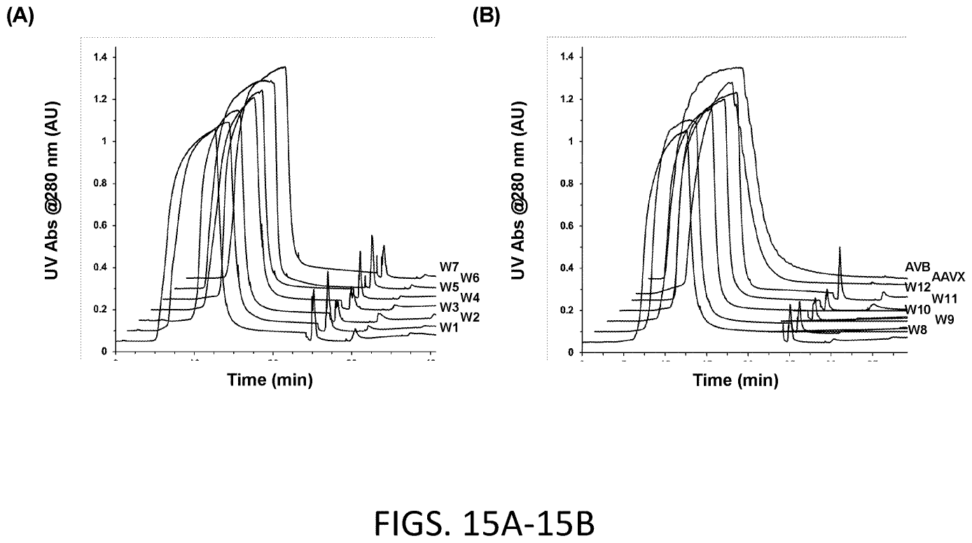

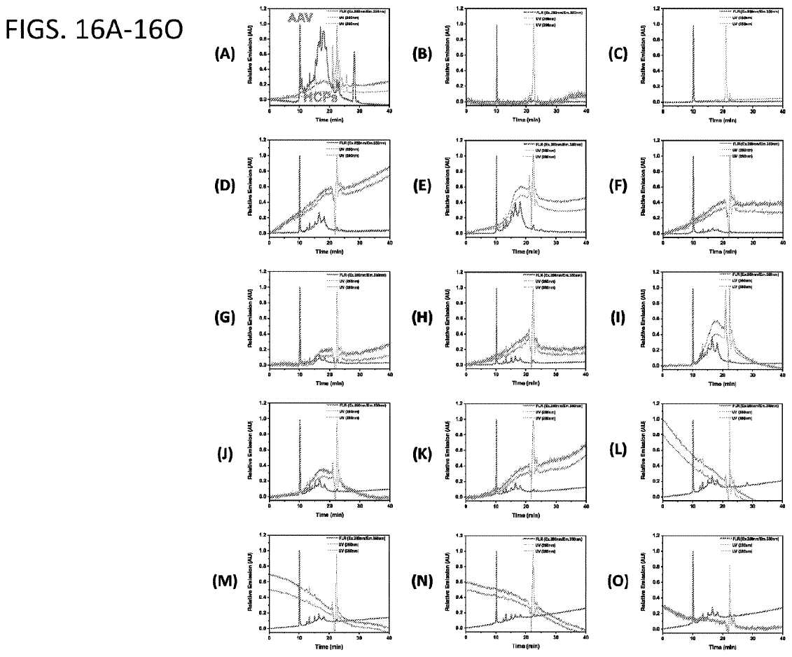

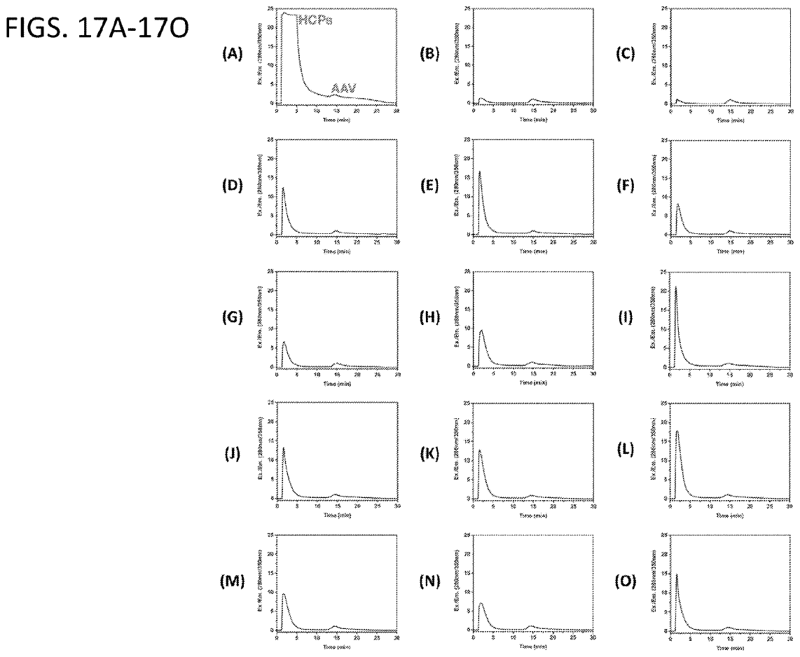

Purification of AAV from HEK 293 cell culture lysate using peptide-functionalized chromatographic resins. Having confirmed the broad targeting activity, high binding capacity, and efficient elution under mild conditions of the peptide ligands, experiments were conducted to evaluate their ability to purify AAV2 from a clarified HEK 293 cell culture lysate. The feedstock was formulated to mimic the harvests typical of the gene therapy industry (AAV2 titer ~1.6·10¹¹ vp/mL; HCP titer ~0.5 mg/mL). A relatively short RT ~3 mins was adopted for the binding step to capitalize on the high AAV binding capacity of the peptide-based adsorbents while attempting to minimize the adsorption of HEK 293 HCPs. The chromatograms of AAV2 purification are collated in FIG. 15, while the size exclusion and steric exclusion chromatography analyses of the collected fractions are reported in FIG. 16 and FIG. 17, respectively; finally, the values of AAV2 yield and logarithmic removal values of HEK 293 host cell proteins (HCP LRV) are summarized in FIG. 4.

The results in FIG. 4 mirror the values of AAV2 capture and release presented in FIG. 2A: (i) product loss in the flow-through and wash fractions oscillated between 1-4%, indicating that the adsorbents maintain their high binding capacity when loaded with complex feedstocks; (ii) the peptide-based adsorbents afforded yields ranging between 40-65%, thus consistently outperforming reference POROS™ CaptureSelect™ AAVX Affinity and AVB Sepharose HP resins; and (iii) 6 out of 12 peptide-based adsorbents afforded values of HCP LRV above 1.8, with IWWHIAKFG-Toyopearl and FWNWHHFKG-Toyopearl resins achieving LRVs of 2.6 and 2.5, respectively—corresponding to a 400-fold and 330-fold reduction of HEK 293 HCPs—thus providing a purification performance comparable to that of commercial reference resins.

The feedstock and elution fractions were further analyzed via size exclusion chromatography (SEC, FIG. 16) and steric exclusion chromatography (SXC, FIG. 17) to evaluate the presence of capsid aggregates and fragments, and visualize the removal of process-related impurities (note: while HEK 293 ELISA assays provide the titer of HCPs, analytical chromatography also reveals the presence of denatured or hydrolyzed HCPs, other non-proteinaceous metabolites, host cell DNA and RNA, and media components); and transduction assay on human epithelial cells (HT1080) to evaluate the recovery of the genetic payload and the infectivity of the purified viruses, respectively.

The SEC and SXC analyses exemplify the complex biomolecular landscape of the feedstock (FIG. 16A and FIG. 17A). Mirroring the ELISA results, the chromatographic profiles of the fractions eluted from POROS™ CaptureSelect™ AAVX Affinity (FIG. 16B and FIG. 17B) and AVB Sepharose HP (FIG. 16C and FIG. 17C) resins highlight the high purity of the eluted AAV2; notably, despite the harsh elution pH, no aggregates were observed in those fractions, potentially owing to the salting-in effect provided by the salt composition and concentration of the respective elution buffers. The viruses eluted from the peptide-based adsorbents are accompanied by some impurities, although the chromatographic profiles of the eluted fractions from IWWHIAKFG- (~205-fold reduction of impurities based on the chromatographic area), FWNWHHFKG- (~160-fold), FWWAAFFKG- (185-fold), FFNFFKG- (135-fold), and FNHFFIG-Toyopearl (145-fold) resins—in line with the results of the ELISA kits—demonstrate the excellent purification activity of the selected peptide ligands.

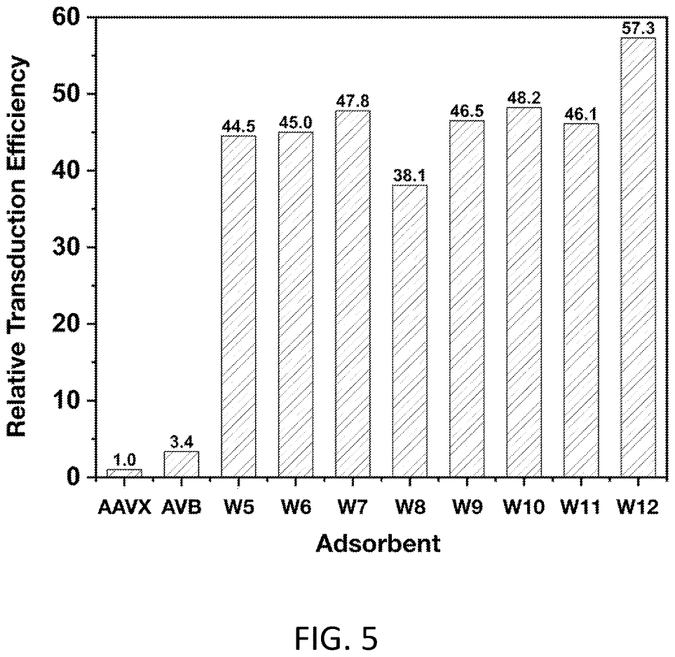

Together with yield and purity, the transduction activity of the eluted viral vectors—namely, their ability to effectively deliver their gene payload to the target cells—is a critical parameter that defines the quality of the purification process. Unlike antibody-based therapeutics, whose biomolecular stability allows leveraging significant variations in buffer conductivity and pH to control their adsorption to and elution from the affinity resins, AAVs are significantly more susceptible to loss of activity driven by bioprocess conditions. Commercial affinity resins for AAV purification, however, mandate product elution under extremely acidic pH, varying between 2-3 depending upon serotype and desired elution yield (>50%) and titer. Conversely, the peptide-based adsorbents presented in this work afford comparable elution performance under significantly milder conditions (1M MgCl₂ in the 20 mM Bis-Tris buffer at pH 6.0). Accordingly, experiments were conducted to resolve the transduction activity of the AAV2 purified using the peptide-based adsorbents vs. the reference AVB Sepharose HP, and CaptureSelect™ AAVX Affinity resins on human epithelial (HT1080) cells. The transgene encapsidated in the model AAV2 utilized in this study encodes for green fluorescence protein (GFP), thus allowing facile quantification of transduction activity via fluorescence flow cytometry. The results collated in FIG. 5 corroborate the claim that elution under mild conditions affords a product with superior transduction activity: the AAV2 purified using peptide-based adsorbents transduced in fact a remarkably higher amount of HT1080 cells, up to 20- to 60-fold more than the AAV2 eluted from commercial affinity resins.

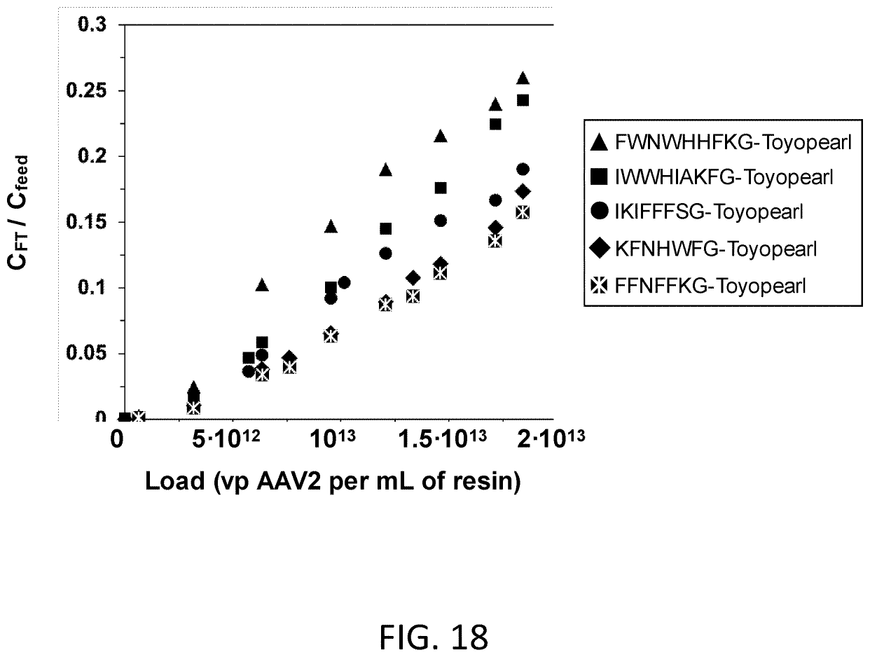

Upon comparing the bind-and-elute performance of the various peptides, experiments were conducted to measure the dynamic binding capacity (DBC_{10%}) of selected adsorbents KENHWFG, IWWHIAKFG, FWNWHHFKG, IKIFFFFSG, and FFNFFKG-Toyopearl resins (FIG. 18). All measurements were conducted at the residence time (RT) of 3 min, which is recommended for both POROS™ AAVX and AVB Sepharose resins. The values of DBC_{10%} reported in Table 4 met the value of 10¹³ viral particles per mL of resin—regarded by the community as the standard for affinity resins intended for AAV purification—thus supporting the adoption of peptides ligands for the product capture step in virus biomanufacturing.

TABLE 4
Values of dynamic AAV2 binding capacity (DBC_{10%}) of peptide-
functionalized resins loaded with a feedstock containing AAV2
in the HEK293 lysate at the titer of 3.2 · 10¹¹ vp/mL
in PBS pH 7.4 at residence time (RT) of 3 mins.

| Resin | DBC_{10%} (vp AAV per mL of resin) |
|---|---|
| KFNHWF-Toyopearl | 1.33 · 10¹³ |
| IWWHIAKF-Toyopearl | 9.53 · 10¹² |
| FWNWHHFK-Toyopearl | 8.75 · 10¹² |
| IKIFFFFS-Toyopearl | 1.02 · 10¹³ |
| FFNFFK-Toyopearl | 1.40 · 10¹³ |

What is claimed is:

1. A composition for purifying an adeno-associated virus (AAV) from a biological fluid comprising:
(a) at least one peptide ligand that is at least six amino acids in length comprising the amino acid sequence X₁-X₂-X₃-X₄-X₅-X₆ (SEQ ID NO: 1), wherein X₁ is F, S, K, or W; X₂ is F, W, N, or K; X₃ is N, F, E, A, I, or W; X₄ is F, I, H, or K; X₅ is F, I, N, or W; and X₆ is K, F, I, S or A; and/or
(b) at least one peptide ligand that is at least eight amino acids in length comprising the amino acid sequence X₁-X₂-X₃-X₄-X₅-X₆-X₇-X₈ (SEQ ID NO: 2), wherein X₁ is F, I, or N; X₂ is W, K, I, or A; X₃ is N, A, W, I, or F; X₄ is W, A, H, F, S or K; X₅ is H, F, A, I, or K; X₆ is H, F, A, or I; X₇ is F, K, W, S, or I; and X₈ is K, F, S, or I.

2. The composition of claim 1, wherein the AAV is selected from the group consisting of AAV1, AAV2, AAV3, AAV4, AAV5, AAV6, AAV7, AAV8, AAV9, and AAVrh10.

3. The composition of claim 1 or claim 2, wherein the AAV is a recombinant AAV (rAAV).

4. The composition of any one of claims 1 to 3, wherein the at least one peptide ligand from (a) and/or (b) binds a site on the AAV capsid.

5. The composition of claim 4, wherein the peptide's binding site on the AAV capsid is a conserved region of the AAV capsid and located at:
(i) an interface of VP1-VP2 virion proteins, or an interface of VP1-VP3 virion proteins, or an interface of VP2-VP3 virion proteins; or
(ii) an surface of the virion protein VP1, VP2, or VP3.

6. The composition of any one of claims 1 to 5, wherein the at least one peptide ligand from (a) and/or (b) comprises an isoelectric point from about 6.5 to about 12.5.

7. The composition of any one of claims 1 to 6, wherein the at least one peptide ligand from (a) and/or (b) comprises a polarity value from about 5.0 to about 8.0.

8. The composition of any one of claims 1 to 7, wherein the at least one peptide ligand from (a) and/or (b) exhibits a disassociation constant (K_{D}) less than or equal to about 10⁻⁴ M at pH 7.4.

9. The composition of any one of claims 1 to 7, wherein the at least one peptide ligand from (a) and/or (b) exhibits a disassociation constant (K_{D}) higher than or equal to about 10⁻⁴ M at a pH that is at least 6.0.

10. The composition of any one of claims 1 to 9, wherein the at least one peptide ligand from (a) and/or (b) exhibits a dynamic binding capacity (DBC_{10%}) of at least 5×10¹² vp/mL of resin.

11. The composition of any one of claims 1 to 10, wherein the composition comprises at least one peptide from (a) and at least one peptide ligand from (b).

12. The composition of any one of claims 1 to 11, wherein the at least one peptide ligand from (a) comprises:
(i) an amino acid sequence having at least 80% sequence identity with SEQ ID NO: 3;
(ii) an amino acid sequence having at least 80% sequence identity with SEQ ID NO: 4;
(iii) an amino acid sequence having at least 80% sequence identity with SEQ ID NO: 5;
(iv) an amino acid sequence having at least 80% sequence identity with SEQ ID NO: 6;
(v) an amino acid sequence having at least 80% sequence identity with SEQ ID NO: 7;
(vi) an amino acid sequence having at least 80% sequence identity with SEQ ID NO: 8;
(vii) an amino acid sequence having at least 80% sequence identity with SEQ ID NO: 9;
(viii) an amino acid sequence having at least 80% sequence identity with SEQ ID NO: 10; and/or
(ix) an amino acid sequence having at least 80% sequence identity with SEQ ID NO: 11.

13. The composition of any one of claims 1 to 11, wherein the at least one peptide ligand from (a) comprises:
(i) an amino acid sequence comprising SEQ ID NO: 3;
(ii) an amino acid sequence comprising SEQ ID NO: 4;
(iii) an amino acid sequence comprising SEQ ID NO: 5;
(iv) an amino acid sequence comprising SEQ ID NO: 6;
(v) an amino acid sequence comprising SEQ ID NO: 7;
(vi) an amino acid sequence comprising SEQ ID NO: 8;
(vii) an amino acid sequence comprising SEQ ID NO: 9;
(viii) an amino acid sequence comprising SEQ ID NO: 10; and/or
(ix) an amino acid sequence comprising SEQ ID NO: 11.

14. The composition of any one of claims 1 to 13, wherein the composition comprises:
(i) at least two peptide ligands selected from SEQ ID NOs: 3-11;
(ii) at least three peptide ligands selected from SEQ ID NOs: 3-11;
(iii) at least four peptide ligands selected from SEQ ID NOs: 3-11;
(iv) at least five peptide ligands selected from SEQ ID NOs: 3-11;
(v) at least six peptide ligands selected from SEQ ID NOs: 3-11;
(vi) at least seven peptide ligands selected from SEQ ID NOs: 3-11;
(vii) at least eight peptide ligands selected from SEQ ID NOs: 3-11; or
(viii) all nine peptide ligands of SEQ ID NOs: 3-11.

15. The composition of any one of claims 1 to 14, wherein the at least one peptide ligand from (b) comprises:
(i) an amino acid sequence having at least 80% sequence identity with SEQ ID NO: 12;
(ii) an amino acid sequence having at least 80% sequence identity with SEQ ID NO: 13;
(iii) an amino acid sequence having at least 80% sequence identity with SEQ ID NO: 14;
(iv) an amino acid sequence having at least 80% sequence identity with SEQ ID NO: 15;
(v) an amino acid sequence having at least 80% sequence identity with SEQ ID NO: 16;
(vi) an amino acid sequence having at least 80% sequence identity with SEQ ID NO: 17;
(vii) an amino acid sequence having at least 80% sequence identity with SEQ ID NO: 18;
(viii) an amino acid sequence having at least 80% sequence identity with SEQ ID NO: 19; and/or
(ix) an amino acid sequence having at least 80% sequence identity with SEQ ID NO: 20.

16. The composition of any one of claims 1 to 14, wherein the at least one peptide ligand from (b) comprises:
(i) an amino acid sequence comprising SEQ ID NO: 12;
(ii) an amino acid sequence comprising SEQ ID NO: 13;
(iii) an amino acid sequence comprising SEQ ID NO: 14;
(iv) an amino acid sequence comprising SEQ ID NO: 15;
(v) an amino acid sequence comprising SEQ ID NO: 16;
(vi) an amino acid sequence comprising SEQ ID NO: 17;
(vii) an amino acid sequence comprising SEQ ID NO: 18;
(viii) an amino acid sequence comprising SEQ ID NO: 19; and/or
(ix) an amino acid sequence comprising SEQ ID NO: 20.

17. The composition of any one of claims 1 to 16, wherein the composition comprises:
(i) at least two peptide ligands selected from SEQ ID NOs: 12-20;
(ii) at least three peptide ligands selected from SEQ ID NOs: 12-20;
(iii) at least four peptide ligands selected from SEQ ID NOs: 12-20;
(iv) at least five peptide ligands selected from SEQ ID NOs: 12-20;
(v) at least six peptide ligands selected from SEQ ID NOs: 12-20;
(vi) at least seven peptide ligands selected from SEQ ID NOs: 12-20;
(vii) at least eight peptide ligands selected from SEQ ID NOs: 12-20; or
(viii) all nine peptide ligands of SEQ ID NOs: 12-20.

18. The composition of any one of claims 1 to 17, wherein the at least one peptide ligand comprises a linker.

19. The composition of claim 18, wherein the linker is bound to the C-terminus of the peptide ligand, and wherein the linker comprises a Glyₙ or a [Gly-Ser-Gly]ₘ, wherein 6≥n≥1 and 3≥m≥1.

20. The composition of any one of claims 1 to 19, wherein the at least one peptide ligand is bound to a solid support.

21. The composition of claim 20, wherein the solid support comprises a non-porous or porous particle, a membrane, a plastic surface, a fiber or a woven or non-woven fibermat, a hydrogel, a microplate, and/or a microfluidic device.

22. The composition of claim 13, wherein the solid support comprises polymethacrylate, polyolefin, polyester, polysaccharide, iron oxide, silica, titania, and/or zirconia.

23. The composition of any one of claims 1 to 22, wherein the biological fluid is a cell culture fluid.

24. The composition of any one of claims 1 to 22, wherein the biological fluid comprises a supernatant and/or a cellular lysate.

25. The composition of claim 23 or claim 24, wherein the biological fluid is derived from CHO cells.

26. The composition of claim 25, wherein the CHO cells are selected from the group consisting of: CHO-DXB11 cells, CHO-K1 cells, CHO-DG44 cells, and CHO-S cells, or any derivatives or variants thereof.

27. The composition of claim 23 or claim 24, wherein the biological fluid is derived from HEK cells.

28. The composition of claim 27, wherein the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof.

29. The composition of claim 23 or claim 24, wherein the biological fluid is derived from a virus production cell line.

30. The composition of claim 29, wherein the virus production cell line is selected from the group consisting of MDCK-S, MDCK-A, Vero cells, LLC-MK2D, PER.C6, EB66, AGE1.CR cells, Spodoptera frugiperda (Sf9) cells, and HeLa cells, or any derivatives or variants thereof.

31. An adsorbent comprising the composition of any of claims 1 to 30.

32. A method of purifying an adeno-associated virus (AAV) from a biological fluid, the method comprising:
contacting the composition comprising the at least one peptide ligand of any one of claims 1 to 30, or the adsorbent of claim 31, with a biological fluid comprising the AAV, wherein the at least one peptide ligand binds the AAV; and
eluting the AAV from the at least one peptide ligand.

33. The method of claim 32, wherein the elution is performed at pH from about 6.0 to about 7.5.

34. The method of claim 32 or claim 33, wherein the method further comprises a washing step before eluting the AAV from the at least one peptide ligand.

35. The method of any one of claims 32 to 34, wherein the method results in at least a 50% yield for the AAV.

36. The method of any one of claims 32 to 35, wherein the method produces at least an 80-fold reduction in host cell proteins.

37. An adeno-associated virus (AAV) purified using the method of claim 32, wherein the AAV exhibits at least 50% transduction activity.


SEQUENCE LISTING (20 sequences)

SEQ ID NO: 1-2 - skipped (2 entries)

SEQ ID NO: 3 - protein, 6 aa, Synthetic construct
FFNFFK

SEQ ID NO: 4 - protein, 6 aa, Synthetic construct
SWFIIF

SEQ ID NO: 5 - protein, 6 aa, Synthetic construct
KWFIIF

SEQ ID NO: 6 - protein, 6 aa, Synthetic construct
FNEFFI

SEQ ID NO: 7 - protein, 6 aa, Synthetic construct
WKAHNK

SEQ ID NO: 8 - protein, 6 aa, Synthetic construct
WWIKIS

SEQ ID NO: 9 - protein, 6 aa, Synthetic construct
KFNHWF

SEQ ID NO: 10 - protein, 6 aa, Synthetic construct
KWWIWA

SEQ ID NO: 11 - protein, 6 aa, Synthetic construct
KWWHWA

SEQ ID NO: 12 - protein, 8 aa, Synthetic construct
FWNWHHFK

SEQ ID NO: 13 - protein, 8 aa, Synthetic construct
IWAWFHFF

SEQ ID NO: 14 - protein, 8 aa, Synthetic construct
FWWAAFFK

SEQ ID NO: 15 - protein, 8 aa, Synthetic construct
IWWHIAKF

SEQ ID NO: 16 - protein, 8 aa, Synthetic construct
IKIFFFFS

SEQ ID NO: 17 - protein, 8 aa, Synthetic construct
NWAWFIWK

SEQ ID NO: 18 - protein, 8 aa, Synthetic construct
FIFSKFFI

SEQ ID NO: 19 - protein, 8 aa, Synthetic construct
IAFKKISI

SEQ ID NO: 20 - protein, 8 aa, Synthetic construct
IAFKKIII